(19) United States
(12) Patent Application Publication
(10) Pub. No.: US 2010/0152687 A1
(43) Pub. Date: Jun. 17, 2010

(54) Feminine undergarment

(76) Inventor: Antonella F. Carlozzi, Hallandale, FL (US)

Correspondence Address: BUCHANAN, INGERSOLL & ROONEY PC, POST OFFICE BOX 1404, ALEXANDRIA, VA, 22313-1404, US

(21) Appl. No.: 12/654,241

(22) Filed: Dec. 15, 2009

Related U.S. Application Data
(60) Provisional application No. 61/138,343, filed on Dec. 17, 2008.

(51) Int. Cl. A61F 13/15; A61F 13/45; A61B 19/02

(52) U.S. Cl. 604/359; 604/367; 604/385.01; 604/387; 604/378; 206/440

(57) ABSTRACT

An undergarment comprises a strapless, coreless web sized and configured between an anterior end and a posterior end to cover, in use, at least the vulva of an adult female wearer. The undergarment is pre-adherent to the wearer, e.g., having pre-applied adhesive or pre-applied suction material on the body side of the web, suitable for contact with human skin and sufficiently adherent to removably hold the undergarment in place during use. In another aspect, a feminine undergarment comprises such pre-adherent strapless, coreless web with a removable protective cover over at least the adhesive. In another aspect, a package of feminine undergarments comprises outer packaging containing at least multiple of such feminine undergarments and optionally inner packaging for each of the undergarments individually.

### PRIORITY CLAIM

This application claims priority to U.S. Provisional Application Ser. No. 61/138,343, filed Dec. 17, 2008, and entitled, Feminine Undergarment, the entire disclosure of which is hereby incorporated by reference.

### INTRODUCTION

The present invention provides feminine undergarments, more specifically, feminine undergarment which are suitable for wearing in lieu of briefs, panties or thong underwear, to achieve a desirable appearance.

### BACKGROUND

It is often desirable in women's fashion to achieve a sleek or smooth appearance with close fitting pants, skirts, dresses or other such garments. This is especially important for high fashion women's clothing. It is often particularly desirable to have no visible signs of underwear to disrupt the smooth appearance, e.g., to have no visible “panty lines” showing through the pants, skirt, or other outer garment. This has often proven to be difficult or impossible to achieve, especially with certain “body conscious” garments formed of thin or clingy fabric or the like. The thickness of even sheer underwear often “reads through” the outer garment. Even if thong underwear is worn in place of more traditional briefs or panties, the presence of the thong underwear can often be seen through the pants, skirt, etc., disrupting the desired appearance. Further, the upper portion of thong underwear and other more traditional underwear may undesirably show above the top of a low cut skirt or pants. Also, G-string and thong underwear and certain other such high fashion undergarments may provide inadequate coverage for some women, potentially causing discomfort and hygiene concerns. While wearing no undergarment at all can achieve a desired smooth appearance, it also can be unhygienic, and many women find it uncomfortable. In addition, feminine underwear can in some circumstances contribute to the spread of infection from clothing to clothing. Laundering underwear is not always effective to prevent such spread of infection, and a more hygienic solution is desirable for such circumstances.

It is an object of this invention to provide feminine undergarments which are suitable for wearing with or in lieu of briefs, panties, G-strings or thong underwear. It is an object of at lease certain exemplary embodiments of the feminine undergarments disclosed here to solve some or all of the fashion, modesty and health concerns mentioned above. At least certain embodiments of the feminine undergarments disclosed here serve other purposes as well. Additional objects and advantages of all or various embodiments of the feminine undergarment disclosed here will be apparent to those of ordinary skill in the art given the benefit of the disclosure and detailed discussion, below.

### SUMMARY

In accordance with a first aspect of the invention, feminine undergarments are disclosed for wearing instead of or in addition to more traditional briefs, panties, G-strings, thong underwear etc. Feminine undergarments disclosed here comprise a strapless, coreless web. The web of the undergarment is pre-adherent to the skin of the wearer. That is, the undergarment, packaged for storage and/or shipment, sale, etc., carries pre-applied adhesive on the body side of the web (the side contacting the wearer's vulva and/or skin surrounding the vulva), comprises a web that is inherently sufficiently adhesive to skin (alone or cooperatively with adhesive, imprinting, grooves, ridges, and/or suction) or is otherwise pre-adherent to the skin of the wearer so as to remain in position covering the vulva.

As further described, below, the “web” can be any fabric, film, mesh, molded or otherwise formed or unformed panel or piece (including multi-piece) material or the like suitable to cover at least, and not necessarily more than, the wearer's vulva and to carry pre-applied adhesive suitable for direct contact with the wearer's skin to hold the web in place on the wearer or to be otherwise pre-adherent.

In certain exemplary embodiments pre-applied adhesive is employed which is sufficiently adherent to skin to removably hold the undergarment in place covering at least the female wearer's vulva.

The undergarment, including the web, pre-applied adhesive and any other components, is suitable for contact with human skin, e.g., short term contact or, in some embodiments or versions of the undergarments even for prolonged contact. In certain exemplary embodiments of the feminine undergarments disclosed here, the pre-applied adhesive is employed that is suitable for contact with the skin surrounding the vulva. In certain embodiments the pre-applied adhesive is suitable for contact with some or all of the vulva itself. For example, as further discussed below, the adhesive may in some versions of the disclosed undergarments be suitable to contact and adhere to the sensitive tissues and mucus membranes of the vulva. The undergarments in certain exemplary embodiments directly contact only the vulva. In other embodiments the undergarments contact the vulva and surrounding skin. In other embodiments the undergarments contact surrounding skin and span over the vulva without directly contacting it.

Certain exemplary embodiments of the undergarments disclosed here are configured to extend rearward to or beyond the anus. In some but not all such embodiments the web or an extension therefrom extends in the gluteal crease to or beyond the anus. In some but not all embodiments the web or an extension therefrom is in direct contact with the anus and/or skin surrounding the anus. In some but not all embodiments the web or an extension therefrom contacts the buttocks so as to span over the anus area, optionally without directly contacting the anus.

The undergarment optionally provides sealing contact with the wearer. In certain exemplary embodiments the undergarment provides sealing contact with the vulva and/or the skin surrounding the vulva so as to form a temporary, full circumference seal over the vulva. Certain, but not all embodiments of the undergarments also provide sealing contact with the anus and/or the skin surrounding the anus so as to form a temporary, full circumference seal over the anus.

The undergarment optionally may fill into areas of the wearer. In certain exemplary embodiments the undergarment provides at least some “fill-in” function of the wearer's creases, anus, vulva, and/or other natural or artificial areas. Some exemplary embodiments may form a temporary, full circumference fill-in of at least some of the vulva. Certain, but not all embodiments of the undergarments also provide fill-in contact with the anus and/or the skin surrounding the anus so as to form a temporary, full circumference fill-in at least over some of the anus.

The undergarment optionally may mold to and/or with the wearer. In certain exemplary embodiments the undergarment provides at least some molding contact with the vulva and/or the skin surrounding the vulva so as to form a temporary, full circumference mold over at least some of the vulva. Certain, but not all embodiments of the undergarments also provide molding contact with the anus and/or the skin surrounding the anus so as to form a temporary, full circumference mold over and/or in at least some of the anus.

The undergarment optionally may have compatibility with other applications to and/or in the wearer's vulva. In certain exemplary embodiments the undergarment provides at least some slitting, perforation, other opening or application for an opening, and/or a combination of two or more of these. The compatibility aspect may be part of the function of the main web, a secondary and/or other web other than the main web, adhesive, and/or both. Certain exemplary devices may have a removable interior web, slitting at the periphery of at least one side of the undergarment, and/or any other application as to facilitate and allow for insertion, removal, and/or any other type of compatibility with external and/or internal devices, objects, etc. (such as suppositories, tampons, catheters, etc). The undergarment optionally may have compatibility with other applications to the wearer's anus and/or ostomy openings. Certain, but not all embodiments of the undergarments also provide compatibility with other applications to and/or in the wearer's anus so as to allow for insertion and/or removal of objections (i.e. suppositories, thermometers, etc). It is understood that any part of the above applications may be removable and/or replaceable.

The feminine undergarment has an anterior end for wearing to the front, a posterior end for wearing to the back, a body side to contact the wearer (i.e., to be worn facing and typically in contact with at least the vulva or surrounding skin) and an outside or outer side facing generally downward away from the wearer's groin or torso.

Various different embodiments of the feminine undergarments are suitable for one or more of several different purposes, including, for example, fashion (e.g., to provide a smooth or sleek under clingy or tight clothing), modesty (e.g., in lieu of wearing no undergarment), hygiene (e.g., to protect the vulva or to cover it to control the spread of a vaginal or other infection from clothing to clothing, etc.), bio-occlusion or semi-occlusion (e.g., to cover the vulva to prevent or reduce odor), and other purposes.

Certain exemplary embodiments of the feminine undergarments disclosed here are edible. Certain edible exemplary embodiments may be comprised of the following: pectin, starch, -gelatin, low-sugar gelatin, sugar-free gelatin, seaweed, agar-agar, xanthan gum, carrageenan, natural fibers, corn fibers, carnauba wax, potato starch, beeswax, and/or a combination of any of these or other components.

Certain exemplary embodiments of the feminine undergarments disclosed here are disposable, rendering them particularly useful for travel, for one-time use to control the spread of a vaginal infection from clothing to clothing, and for other purposes. Different and additional uses will be apparent in view of this disclosure to those skilled in the art and/or to women wearing the undergarments disclosed here.

The web of the feminine undergarments disclosed here is configured and sized longitudinally between the anterior end and the posterior end and laterally from side to side between the wearer's thighs to cover, in use, a human female's vulva (meaning the entire vulva). In certain exemplary embodiments it is configured and sized longitudinally and laterally to cover at least an adult female wearer's vulva. In certain other exemplary embodiments it is configured and sized longitudinally and laterally to cover at least the vulva of a pre-adult, e.g., an adolescent or child. It should be understood that reference here to dimensions suitable to cover a female wearer's vulva means sufficiently large for at least the 75th percentile, i.e., sufficiently large to cover the vulva of at least 75% of adult female wearers in a particular region. Thus, embodiments for an adult female can be large enough to cover the vulva in at least 75% of female adults (i.e., ages 18 years and above) in the United States. Certain such undergarments will be sufficiently large for the 75^{th} percentile and smaller, while other such undergarments will be sufficiently large for even larger women. Likewise, embodiments for a pre-adult are large enough to cover the vulva in at least 75% of female pre-adults (i.e., ages less than 18 years old) in the United States. Embodiments for adolescents are large enough to cover the vulva in at least 75% of female adolescents. Embodiments for any particular age range of children are large enough to cover the vulva in at least 75% of female children in the United States within that age range, e.g., 75% of female children in the United States below the age of 13, or 75% of female children in the United States within the range of ages 8 to 13, ages 3 and older, ages 3-13, ages 3-8, etc. Certain dimensions for various exemplary (i.e., non-limiting) embodiments or examples of the feminine undergarments disclosed here are further discussed below.

In certain exemplary embodiments the web is sufficiently large for most or all users. In certain exemplary embodiments the distribution of adhesive on the body-side and the size and configuration of the web are suitable for the web to be trimmed by the user prior to use, e.g., to be cut, torn or otherwise adjusted to the size needed by that user. In certain exemplary embodiments the web includes one or more printed lines, perforation lines or other guide(s) for size-adjustment of the web by the user. For example, the web may have one or more guide lines for size adjustment, e.g., lines of closely spaced perforations extending along and generally parallel to (i.e., uniformly spaced from) the peripheral edge of the web. Such guide lines can be used to guide the required or optional removal of a peripheral portion of the web by hand tearing, cutting with scissors, etc. to reduce the size of the web. Similarly, the web may have one or more lines marked on the upper surface of the web, the lower surface of the web or both, extending along and generally parallel to the periphery of the web, whereby a peripheral portion of the web may optionally be removed by cutting with scissors or, for at least certain suitable web materials, hand tearing, etc. to reduce the size of the web. In certain exemplary embodiments the web has multiple such lines of perforations or other guides to facilitate reduction in the size of the web to any of multiple smaller sizes. The web optionally may have adhesive tabs which are optionally removable in the same or other fashion by the user. Certain exemplary embodiments may have perforations to separate web and/or adhesive sections (i.e. the web and/or adhesive covering the vulva may be separated from the web and/or adhesive covering the anus).

In certain exemplary embodiments the web, adhesive, protective lining and/or a combination of two or more may have a tab, grip strip, and/or any other acceptable function for separating at least some of the undergarment from the envelope, at least some of the web covering the vulva from the web covering the anus, at least some of the adhesive from it's protective lining, at least some of the undergarment from the wearer, at least some the main web from secondary web(s), at least some of the web or any part of the web from the adhesive, at least some of the web from the wearer, at least some of the adhesive from the wearer and/or any combination of one or more these. The grip strip, tab, and/or other suitable means may be located on any portion of the web, adhesive, protective lining, encased envelop, and/or any combination of these.

In accordance with another aspect of the invention, a feminine undergarment comprises a strapless, coreless web, pre-applied adhesive on the body side of the web suitable for contact with human skin, e.g., prolonged contact with the sensitive tissues of the vulva, and sufficiently adherent to removably hold the undergarment in place during use covering a female wearer's vulva, and a removable protective layer covering at least the adhesive and removable by the wearer prior to use of the undergarment. In certain exemplary embodiments the removable protective layer comprises a film, sheet or the like. In certain exemplary embodiments the removable protective layer covers all or only a portion of the undergarment and is formed of a single piece or multiple pieces of film or other sheet-like material such as paper, coated paper, plastic, etc. Any or all of the protective layer may be clear, translucent or transparent. In certain exemplary embodiments the removable protective layer may comprise an open or sealed, e.g., hermetically sealed, envelope containing the undergarment, e.g., a disposable envelope. The envelope may be formed, for example, of paper, clear, translucent, transparent or opaque plastic material, e.g., colored or non-colored cellophane, etc.

In accordance with another aspect of the invention, feminine undergarments comprise a strapless, coreless web and suction means for holding the web in position covering at least the wearer's vulva, e.g., suction material on a body side of the web, suitable for contact, e.g., short term or prolonged contact, with human skin, and suitable during use (either alone or in combination with adhesive) to removably hold the undergarment in place by maintaining a suction against the wearer's skin, covering at least the wearer's vulva. In certain exemplary embodiments the suction material is sufficiently soft and pliable, e.g., malleable or the like, to conform generally to the skin contours of the wearer, and so configured and sufficiently resilient, e.g., with a degree of shape memory, to hold suction against the wearer's skin. The suction may in certain exemplary embodiments be a single area of suction to the skin of the wearer. Such suction area may, for example, span the vulva or not. A single suction area or multiple suction areas may, instead or in addition, be adjacent to the vulva (e.g., the perineum) or remote from the vulva (e.g., one or more areas of the buttocks, one or more areas of the mons veneris, one of more areas of the inner thighs, and/or other areas). In certain exemplary embodiments the suction material is grooved, dimpled or other configuration operative to form multiple smaller areas of suction against the wearer. As with the other aspects disclosed above, the feminine undergarments of this aspect have an anterior end for wearing to the front, a posterior end for wearing to the back, a body side to contact the wearer (i.e., to be worn facing and typically in contact with at least the vulva and/or surrounding skin) and an outside or outer side facing generally downward away from the wearer's groin or torso.

In accordance with another aspect of the invention, a package comprises multiple feminine undergarments and associated packaging, i.e., packaging containing or otherwise holding at least such feminine undergarments. The packaging is openable, e.g., releasable, by the consumer and, in certain exemplary embodiments, is re-sealable or otherwise re-closable to contain any remaining undergarments after removal of one or more of the undergarments. The multiple feminine undergarments include at least one or more of the feminine undergarments disclosed above, each optionally with a removable protective layer protecting pre-applied adhesive. A package in accordance with this aspect of the disclosure may contain two of the undergarments or more, e.g., three, four, five, six, seven, eight, nine, ten, a dozen, fifteen, or more.

Various non-limiting examples of the feminine undergarments disclosed here are described in more detail below. Those of ordinary skill in the art will appreciate from the disclosure above and the detailed description below, that the feminine undergarments disclosed here provide a useful option to women. All or at least certain embodiments of the feminine undergarments are useful especially, e.g., for wearing with high fashion clothing, such as close fitting pants, skirts, dresses or other such garments, particularly when the woman desires to have no visible signs of underwear to disrupt a smooth appearance, e.g., to have no visible “panty lines” showing through the pants, skirt, or other outer garment. All or at least certain embodiments of the feminine undergarments, especially disposable versions are useful to control the spread of infection from clothing to clothing and for other such hygiene purposes. Additional feature and advantages of the feminine undergarments disclosed here, or at least of certain exemplary embodiments of such feminine undergarments, will be appreciated from the following discussion.

### BRIEF DESCRIPTION OF THE DRAWINGS

The discussion below refers to the appended drawings showing certain exemplary embodiments of the feminine undergarments disclosed here.

FIG. 1A is a plan view of a first embodiment of the feminine undergarments disclosed here, showing the body side of the undergarment;

FIG. 1B is a section view of the embodiment of FIG. 1A, shown being worn;

FIG. 1C is a perspective view of the embodiment of FIGS. 1A and 1B, shown being worn;

FIGS. 2A and 2B are elevation views of alternative versions of the embodiment of FIG. 1, where FIG. 2A has a mono-layer web and FIG. 2B has a duo-layer web;

FIG. 3A is a plan view of a second, embodiment of the feminine undergarments disclosed here, showing the body side of the undergarment;

FIG. 3B is a section view of the embodiment of FIG. 3A, shown being worn;

FIG. 4 is a plan view of a third embodiment of the feminine undergarments disclosed here, including adhesive tabs, showing the body side of the undergarment;

FIG. 5 is a plan view of a fourth embodiment of the feminine undergarments disclosed here, including adhesive tabs, showing the body side of the undergarment;

FIG. 6A is a plan view of a fifth embodiment of the feminine undergarments disclosed here, showing the body side of the undergarment;

FIG. 6B is a section view of the embodiment of FIG. 6A, shown being worn;

FIG. 7A is a plan view of a sixth embodiment of the feminine undergarments disclosed here, showing the body side of the undergarment;

FIG. 7B is a section view of the embodiment of FIG. 7A, shown being worn;

FIG. 8 is a perspective view of a seventh embodiment of the feminine undergarments disclosed here, having a pre-configured cupped web with shape retention;

FIG. 9 is a section view of the undergarment of FIG. 8, taken through line 9-9 in FIG. 8;

FIG. 10 is a perspective view of an eighth embodiment of the feminine undergarments disclosed here, having a pre-configured saddle-shaped web with shape retention;

FIG. 11 is a section view of the embodiment of FIG. 10, taken through line 11-11 in FIG. 10;

FIG. 12 is a plan view of a star-shaped embodiment;

FIG. 13 is a plan view of another alternative embodiment;

FIG. 14 is a perspective view of an embodiment that is both longitudinally and laterally asymmetrical, having an adhesive perimeter and tabs;

FIG. 15 is a plan view of an alternative embodiment, having an adhesive perimeter and tabs;

FIG. 16 is a plan view of an alternative embodiment, having an adhesive perimeter;

FIG. 17 is a plan view of an alternative embodiment, having an adhesive perimeter and tab;

FIGS. 18A-18C are plan views of alternative embodiments configured for covering the wearer's vulva and anus;

FIGS. 19A and 19B are plan views of alternative embodiments of undergarments in accordance with the present disclosure, having a generally diamond shape;

FIG. 20 is a plan view of an alternative embodiment of the undergarments disclosed here, having a generally tulip shape;

FIG. 21 is a plan view of an alternative embodiment of the undergarments disclosed here, having a generally daisy-petal shape with an extension configured to extend into the gluteal crease of the wearer;

FIG. 22 is a plan view of an alternative embodiment of the undergarments disclosed here, having a generally daisy-petal shape;

FIG. 23 is a plan view of a generally heart-shaped alternative embodiment of the undergarments disclosed here;

FIG. 24 is a plan view of another generally heart-shaped embodiment of the undergarments disclosed here; and

FIG. 25 is a plan view of another embodiment of the undergarments disclosed here;

FIG. 26 shows another embodiment of the feminine undergarments disclosed here;

FIG. 27 shows yet another embodiment of the feminine undergarments disclosed here;

FIG. 28 shows yet another embodiment of the feminine undergarments disclosed here; and

FIG. 29 shows yet another embodiment 375 that is compatible meaning compatible with inserting and/or removing objects into the vulva 376.

FIG. 30A-30C shows another embodiment that suctions to the wearer's genital area and may have a component that is removable, replaceable, insertable or any combination thereof. FIGS. 30B-30C are compatible with inserting and/or removing objects into the vulva 388, 392 respectively.

FIG. 31A-31C shows another embodiment that conforms to the female genital area and anus. These embodiments may have insertable linings, webs, devices, or any combination thereof that extend into the genital area and/or anus 395, 400, respectively. They may also be compatible with inserting and/or removing objects into the genital area and/or anus.

### DETAILED DESCRIPTION OF CERTAIN EXEMPLARY EMBODIMENTS

Regarding directional references, the term “anterior” means the front of the wearer or toward the front of the wearer, i.e., at or toward the wearer's mons pubis or navel, or a portion or periphery of the undergarments disclosed here or of the coreless, strapless web of the undergarments disclosed here, that is worn or intended or configured to be worn to the front. The term “posterior” means the back of the wearer or toward the back of the wearer, i.e., at or toward the wearer's perineum or buttocks, or a portion or periphery of the undergarments disclosed here or of the coreless, strapless web of the undergarments disclosed here, that is worn or intended or configured to be worn to the back. The term “lateral” means side-to-side, such as left- to right of the wearer, e.g., from the inside of one of the wearer's thighs toward the inside of the other thigh. In general, the phrase “body side” refers to the side or surface of the web that contacts or faces the wearer's body or that is intended or configured to be worn against or facing the wearer's body, e.g., the upper surface of the web that contacts the wearer's vulva rather than the opposite surface of the web that in use normally faces downward.

In the following description of certain non-limiting examples of the innumerable alternative embodiments of women's undergarments disclosed here and claimed, the term “coreless” web as used here means that the web does not have the absorbent pad or filler materials of femcare products, i.e., sanitary napkins. The “coreless” web may be absorbent in nature, i.e. sponge, foam, medical foam, seaweed, hydrocolloid, algae, etc. The web may be a mono-layer web or a multi-layer web. In the case of multi-layer webs, different materials or the same may be used to form the different layers. In certain exemplary embodiments the web is all-natural, organic (i.e., grown or produced organically, such as without pesticides, artificial fertilizers, etc.) or both. Further, the entire undergarment optionally is all-natural, organic, or both. In certain exemplary embodiments the web is impregnated or otherwise comprises an active agent as further described elsewhere in this disclosure, e.g., a deodorant, perfume, desiccant powder or other drying agent, anti-perspirant, antibacterial, anti-fungal, probiotic, etc., or a combination of any two or more of them.

Any one or more of the layers of the web of the undergarments disclosed here may be formed of one or a combination of materials. Suitable materials will be readily apparent to those of ordinary skill in the art given the benefit of this disclosure and include numerous commercially available materials. Exemplary suitable materials include, e.g., woven, unwoven, knitted, lace, stitched, pressed, or other fabric or soft cloth, paper, silicone, hydrocolloid, or other suitable material(s) or the like, such as a single layer of natural fiber fabric, optionally organically grown natural fiber, e.g., cotton, flax, or the like, or a single layer of synthetic fiber fabric, e.g., rayon, polyester, nylon, spandex, sponge, foam (e.g. medical foam), lycra or the like, or a single layer of mixed natural and synthetic fiber fabric. Other exemplary materials include resin, twill, canvas, burlap (hessian), silk, felt, wool, polypropylene, denim, leaves, fibers, weave, hemp, and/or a combination of one or more materials. For example, all or a portion of the web may be formed of high twist cotton elastic cloth, e.g., with rubber-based, acrylic or other adhesive, optionally with a feathered edge. All or any portion(s) of any such materials of the web may be perforate or imperforate, porous or nonporous, moisture permeable, semi-permeable or impermeable, opaque, translucent and/or transparent. Correspondingly, all or any one or more portions of the web may be perforate or imperforate, porous or nonporous, moisture permeable or impermeable, opaque, translucent and/or transparent.

All or any portion of any such materials of the web may be coated or uncoated, e.g., coated (including impregnated or infused) with silicone or other polymer, wax, sizing, etc. In certain exemplary embodiments the web or one or more layers of the web may be formed of a nonwoven fabric single coated with a silicone gel adhesive, such as BIOFLEX® RX1248P available with a clear film release liner from Scapa North America (Windsor Connecticut). In certain exemplary embodiments the web or one or more layers of the web may be formed of a copolyester film single coated with a silicone gel adhesive, such as BIOFLEX® RX1267P available with a clear film release liner from Scapa North America (Windsor Connecticut). Alternatively any single layer of the web may optionally be formed of any combination of such materials, e.g., different materials connected to each other along seams or the like to form a mono-layer web. In certain exemplary embodiments of the undergarments disclosed here, both the web and the adhesive comprise silicone material. Optionally, the undergarment is formed entirely or essentially entirely of silicone material(s), and in some such versions is fully washable and reusable. As noted above, certain exemplary embodiments of the undergarments disclosed here are pre-adherent in that they are formed of material that is inherently adhesive to human skin adequately (alone or cooperatively with adhesive, imprinting, grooves, ridges, and/or suction) to hold itself in position on the wearer. Thus, for example, the web may in some such embodiments be formed of an inherently adhesive silicone material. In certain exemplary embodiments such inherently adhesive silicone material may be, e.g., an essentially flat sheet of flexible silicone material forming all or a portion of the web. In certain exemplary embodiments such inherently adhesive silicone material is a preconfigured, molded body of silicone material, e.g., molded to have the outward configuration of a healthy vulva for use as a prosthetic or molded to have a cup configuration with the outward or e.g. molded to cup the vulva with a smooth outward configuration.

As noted above, the web of an undergarment in accordance with this disclosure optionally may be a multi-layer web. For example, the web can be a duo-layer web, a tri-layer web, etc. A multi-layer web can be formed of overlying layers of fabric or other materials. Suitable materials include, e.g., any of those fabrics and other materials mentioned above. The layers may be the same as each other or different from each other. For example, the web may comprise overlying layers of cotton fabric or an inner layer of cotton fabric overlying an outer layer of synthetic fabric, etc. The web may be multi-layer only in certain portions or areas, and mono-layer in other areas. In certain exemplary embodiments all or a portion of the web is duo-layer. In certain exemplary embodiments all or a portion of the web is tri-layer, that is, formed of three layers of material overlapping each other. Suitable materials include, e.g., any of those fabrics mentioned immediately above. In tri-layer versions of the web, any of the three layers may be the same as one or both of the other layers, or each may be different from both other layers. For example, the web may comprise overlying layers of cotton fabric or an inner layer of cotton fabric with two outer layers of synthetic fabric, etc. The web may be tri-layer only in certain portions or areas, and mono-layer or duo-layer in other areas. In multi-layer webs, one layer may be held in position by any suitable technique or combination of techniques. For example, one layer may be held to another by adhesive, hook-and-loop joining, fitting into a peripheral edge treatment, etc. For convenience, unless otherwise stated, a duo-layer web is taken here to be a web that is mostly two layers, optionally with certain areas, e.g., edges, etc., having fewer or more layers. Similarly, a tri-layer web, unless otherwise stated, is taken here to be a web that is mostly three layers, optionally with certain areas having fewer or more layers.

All or a portion of the edge of the web and/or other components of the undergarment optionally may be edge-treated, i.e., hemmed, sewed, taped, serged or otherwise treated to prevent fraying and/or for improved appearance, comfort, shape retention, etc. For example, in a mono-layer web having only a single layer of fabric or material, all or a portion of the edge of that layer optionally is hemmed, sewed, taped, serged or otherwise edge-treated to prevent fraying and/or for improved appearance, comfort, shape retention etc. Similarly, all or a portion of the periphery of one or more layers of a duo-layer or tri-layer web or other multi-layer web may be hemmed, taped, serged or otherwise treated to prevent fraying and/or for improved appearance, comfort, shape retention etc. In a duo-layer web, or tri-layer or other multi-layer web, the layers may be sized and positioned so as to entirely or only partially overlap each other.

Optionally, the adhesive feminine undergarments disclosed here may extend rearwardly into the gluteal crease. That is, in certain exemplary embodiments the web or an extension from the web extends rearward toward, to, in, or past the anus, with or without having pre-applied body-side adhesive on such rearwardly extending gluteal crease extension. If present, the pre-applied adhesive of such gluteal crease extension is sufficiently adherent to human skin to removably hold the gluteal crease portion in place in the gluteal crease during wearing. In certain exemplary embodiments pre-applied adhesive is positioned to cover and adhere to skin tissue fully surrounding the anus. In certain exemplary embodiments the pre-applied adhesive is positioned to cover and adhere to skin tissue including the anus, so as to form a removable, i.e., temporary seal of the anus.

Certain exemplary embodiments of the adhesive feminine undergarments disclosed here may be referred to, for convenience, as minimalist undergarments. Such embodiments are shaped and sized to cover the vulva without extending either laterally or forward of the labia. Optionally, such minimalist embodiments may extend rearward into the gluteal crease, with or without body-side adhesive on the gluteal crease portion of the web.

The layers of a duo-layer web, tri-layer web or other multi-layer web embodiment of the adhesive undergarments disclosed here optionally may only partially overly or overlap each other. That is, they optionally may not be coextensive. Any of the layers may extend beyond the other(s) or not, and one or more layers may carry the body-side adhesive used to hold the undergarment in place on the wearer. In certain exemplary embodiments of the feminine undergarments disclosed here, the inner or body-side layer of the web is smaller than the outer layer(s). For example, the inner layer can be sized to cover just the wearer's vulva and the outer layer(s) sized to extend beyond the vulva, e.g., from the anterior end positioned on the female wearer forward of the vulva to the posterior end positioned at or rearward of the perineum.

Certain non-limiting versions of duo-layer tri-layer and/or other multi-layer embodiments may have a removable and replaceable web portion. For example, certain exemplary embodiments may have an outer layer, inner layer or other portion of the web and/or adhesive which may optionally be removable and replaceable. For example, a portion located to directly contact the vulva and/or a decorative element, etc., is removable from the rest of the web and replaceable by a replacement portion. For example, a fresh replacement portion can be used to replace a removable inside vulva-contacting portion. In certain exemplary embodiments the replaceable portion may optionally be impregnated or otherwise comprise one or more active agents as further described elsewhere in this disclosure, e.g., perfume, cologne, essential oils, flavorant, pharmaceutical, nutriceutical, nutrient, probiotic, anti-viral, viricide, antibacterial, antiperspirant, anti-fungal, antiseptic, deodorant, odor neutralizer or suppressant, anti-microbial, microbicide, anti-itching agent, desiccant powder or other drying agent, cooling agent, and any other suitable agent(s) medically or cosmetically useful, or a mixture of any two or more of them. The replaceable portion may be held in position by any suitable technique or combination of techniques, e.g., adhesive, Velcro® or other hook-and-loop joining, fitting into a peripheral edge treatment, etc. In certain exemplary embodiments the replaceable inside portion may be an absorbent or nonabsorbent non-stick layer, optionally flush to the outer peripheral edge of the web in that area of the web. Also, for example, a decorative portion, e.g., a printed outer layer of the web may be removable and replaceable with a differently decorated replacement portion. A replacement portion may be used in certain exemplary embodiments to provide fresh adhesive areas.

The term “strapless” web as used here means that the web has no strap encircling the torso or the leg on either side, such as the strings or straps of a bikini bottom or those of a thong or G-string undergarment. Even the thinnest straps of such traditional undergarments can present a potential disruption to a desired visual appearance of no panty lines or no panties under the wearer's clothes. It should be understood, however, that certain exemplary embodiments of the feminine undergarments disclosed here may have cut-outs or extensions which in some cases may be similar to thin straps but which do not encircle the torso or the leg on either side. Further, some embodiments of the undergarments disclosed here may have an anus extension that extends from the vulva covering into and/or over the gluteal crease.

The term “pre-applied” as used here in reference to body side adhesive of the undergarments means that the adhesive is on the intended surface area or areas of the body side of the undergarment, in proper position for use of the undergarment. The adhesive is on the undergarment as packaged, such that it is ready to be worn. In certain exemplary embodiments a protective film, e.g., a removable film or sheet on the adhesive and optionally also covering all of body-side of the web or a portion thereof covers the adhesive as the undergarment is packaged. In certain exemplary embodiments the pre-applied adhesive is simply an inherent property of the material of the web, e.g., moldable, flexible silicone materials such as are used for strapless bras that naturally adhere directly to the skin. Such silicone webs can be molded to have a desired vulva form and worn, self-adhesively, directly over the vulva. Such embodiments of the undergarments disclosed here can in some cases serve in the nature of a prosthetic device to enhance the wearer's own shape. In certain exemplary embodiments of the feminine undergarments disclosed here having pre-applied adhesive on the web, the pre-applied adhesive comprises a first adhesive on at least a first area of the body-side surface of the web and a second adhesive, different from the first adhesive, on at least a second surface area of the web. For example, a first adhesive along all or some of the perimeter of the web may be stronger (i.e., more strongly adherent to skin) or less strong than a second adhesive used in the center area of the body-side surface of the web. In other embodiments, a first adhesive along one portion of the perimeter of the web may be stronger than a second adhesive used in another portion of the perimeter of the web. In other embodiments, a first adhesive on an extension of the web in the gluteal crease may be stronger or less strong than a second adhesive used at the vulva. The first adhesive may, for example, be a pressure sensitive adhesive while the second adhesive is an inherently adhesive silicone material. Other suitable combinations of two or more adhesives will be readily apparent to those of ordinary skill in the art given the benefit of this disclosure. Similarly, any suitable one or more pre-applied adhesive and/or inherently adhesive web material may be used in combination with suction of the web and/or adherent grooves, ditches, imprints, ridges, or other adherent applications of the web to the wearer, as discussed elsewhere in this disclosure.

Adhesive may be pre-applied to some or all of the body side of the web. With such pre-applied adhesive, the undergarment in certain exemplary embodiments can be applied onto the body by the wearer or an assistant after removal of any cover, e.g., a disposable sheet or envelope, simply by positioning the undergarment and applying gentle pressure. In certain exemplary embodiments, as discussed further, below, the pre-applied adhesive is a pressure sensitive or contact adhesive, such that the undergarment can be applied by positioning it and applying gentle pressure. In certain exemplary embodiments the adhesive is moisture and/or heat activated, such that the undergarment can be applied by holding it in position for a short time, in some embodiments following pre-wetting of the adhesive with water. For example, thermal adhesive may be used. In certain exemplary embodiments the adhesive is organic and/or all-natural. In certain exemplary embodiments the web is all-natural and/or organic. In certain exemplary embodiments the entire undergarment is organic and/or all-natural.

Suitable adhesives are commercially available and their use in the minimalist undergarments disclosed here will be readily apparent to those of ordinary skill in the art given the benefit of this disclosure. The adhesive may be uniform or non-uniform in thickness over the web. Optionally, the adhesive differs in thickness and/or type from one area of the web to another. In certain exemplary embodiments different adhesives may be used in different portions of the undergarment. For example, a less aggressive adhesive, that is, an adhesive forming a weaker adhesive bond to skin, may be used in direct contact with the vulva or portions of the vulva or with the anus, while more aggressive adhesive, that is, adhesive forming a stronger adhesive bond to skin, may be used in contact with surrounding skin areas, the buttocks, the mons veneris, etc. Likewise, different types of adhesive may be used in different areas, e.g., pressure sensitive adhesive in certain areas, moisture activated adhesive in other areas, etc.

In certain exemplary embodiments the portion of the web having pre-applied adhesive is formed of a material or combination of materials different from the material(s) used for some or all of the rest of the web. Likewise, such adhesive carrying portion(s) of the web may be perforate and the some or all of the rest of the web imperforate, or vise versa.

In certain exemplary embodiments of the feminine undergarments disclosed here, the strapless, coreless web is suitable for contact with human skin, optionally for prolonged contact with human skin, e.g., for contact (for a majority of women) in excess of 1 hour. Certain exemplary embodiments are suitable for contact up to 2 hours. Certain exemplary embodiments are suitable for contact up to 3 hours. Certain exemplary embodiments are suitable for contact up to 4 hours. Certain exemplary embodiments are suitable for contact up to 6 hours or even longer. Certain exemplary embodiments are hypoallergenic, i.e., for a majority of wearers cause no significant adverse dermatological reaction, e.g., no reaction beyond possibly a minor, temporary skin irritation in the area. In certain exemplary embodiments the strapless, coreless web is permeable, that is, the web is permeable to oxygen, moisture and liquid. In certain exemplary embodiments the strapless, coreless web is non-permeable. In certain exemplary embodiments the strapless, coreless web is semi-permeable, that is, the web is permeable to moisture but not to liquid. Certain exemplary embodiments are formed at least in part of such semi-permeable materials as GoreTex®. Certain exemplary embodiments comprise hydrocolloids.

Suitable adhesives include various commercially available adhesives and others whose use will be readily apparent to those of ordinary skill in the art given the benefit of this disclosure. The adhesive employed on certain versions or embodiments of the undergarments disclosed here may be a combination of adhesives, e.g., with different adhesives applied to different areas of the body-side of the web. All or a portion of the adhesive, for example, can be pressure sensitive adhesive. All or a portion of the adhesive, for example, can be moisture activated, etc. All or a portion of the adhesive preferably is compatible with the skin of at least a large percentage of women, including the sensitive areas of or around the vulva. Skin compatibility here means that the adhesive does not cause undue skin irritation and inflammation. In certain exemplary embodiments of the undergarments disclosed here, all or a portion of the adhesive has good adhesion to wet skin. In certain exemplary embodiments all or a portion of the adhesive is compliant with the skin of the wearer, such that good contact is achieved and maintained between the adhesive and the skin. The adhesive should be removable from the wearer without causing an unacceptable level of discomfort, and it will be understood from this disclosure that this will be a function of both adhesive strength and the amount and placement of the adhesive on the web. In certain exemplary embodiments the adhesive is suitable to be removed and reapplied one or more times before disposal of the undergarment, e.g., while the wearer is away from home for part or all of a day or night. In certain exemplary embodiments the adhesive remains sufficiently adhesive after the undergarment is removed, laundered and reapplied one or more times. Exemplary suitable adhesives include high cohesive strength adhesives, such as rubber based adhesives and acrylics. Suitable adhesives having good adhesion to wet skin include at least certain of those disclosed, for example, in WO 98/03208, the disclosure of which is incorporated herein by reference, which discloses skin compatible pressure sensitive adhesives which can adhere to dry or wet skin and which comprise a mixture of hydrophilic (meth)acrylate copolymer containing tertiary amino groups, a hydrophilic (meth)acrylate copolymer containing carboxyl groups, carboxylic acids and a cross-linking system. Suitable adhesives include at least certain of those disclosed in WO-A-95/20634, the disclosure of which is incorporated herein by reference, including a bicontinuous pressure sensitive adhesive having a continuous phase of a hydrophobic pressure sensitive adhesive polymer and a continuous phase of a hydrophilic polymer. Suitable adhesives include at least certain of those disclosed in WO-A-97/05171, the disclosure of which is incorporated herein by reference, including a bicontinuous pressure sensitive adhesive having a continuous phase of a hydrophobic pressure sensitive adhesive polymer and a continuous phase of a hydrophilic polymer, the adhesive having peel adhesion of at least 3 Newtons per 100 mm as measured according to a PSTC-1 Test as defined in the disclosure. Suitable adhesives include at least certain of those disclosed in WO-A-97/24149, the disclosure of which is incorporated herein by reference, including a lipophilic polar pressure sensitive adhesive stated to have enhanced adhesion to greasy skin, the adhesive including a hydrophilic polymer matrix, a polar organic plasticizer and at least 9 wt % of a surfactant having an HLB (hydrophile lipophile balance) value of 10 to 17. It is stated generally that the hydrophilic polymer matrix may be selected from a range of polymers including homo- and copolymers of, for example, (meth) acrylic acid and salts thereof, acrylamide, N-vinyl pyrrolidone and acrylamidopropane sulphonic acid and salts thereof. The adhesive is prepared by polymerization in a homogeneous aqueous mixture. Suitable adhesives include at least certain of those disclosed in U.S. Pat. No. 5,338,490 (Dietz et al), the disclosure of which is incorporated herein by reference, including a two-phase composite pressure sensitive adhesive having discontinuous hydrophobic pressure sensitive adhesive domains within a continuous hydrophilic phase. Suitable adhesives for at least some embodiments of the undergarments disclosed here include also certain adhesives comprising cell adhesion molecules, including natural and/or synthetic proteins.

Certain suitable adhesives have a three dimensional matrix or gel comprising a naturally or synthetically derived polymer which can be physically or chemically crosslinked. The uncrosslinked polymer includes repeating units or monomers derived from vinyl alcohols, vinyl ethers and their copolymers, carboxy vinyl monomer, vinyl ester monomers, esters of carboxy vinyl monomers, vinyl amide monomers, hydroxy vinyl monomers, cationic vinyl monomers containing amines or quaternary groups, N-vinyl lactam monomer, polyethylene oxides, polyvinylpyrrolidone (PVP), polyurethanes, acrylics such as methyl acrylate, 2-hydroxyethyl methacrylate, methoxydiethoxyethyl methacrylate and hydroxydiethoxyethyl methacrylate, acrylamides, and sulphonated polymers such as acrylamide sulphonated polymers for example 2 acrylamido methylpropane sulphonic acid and acrylic (3-sulphopropyl) ester acid, and mixtures thereof. Also acrylonitrile, methacrylamide, N,N,-dimethylacrylamide (NNDMA), acrylic esters such as methyl, ethyl and butyl acrylates. Alternatively, the uncrosslinked polymer may be a homopolymer or copolymer of a polyvinyl ether, or a copolymer derived from a half ester of maleic ester. Similarly any other compatible monomer units may be used, such as, for example, polyvinyl alcohol and polyacrylic acid or ethylene and vinyl acetate. Alternatively, the polymers may be block copolymer thermoplastic elastomers, such as ABA block copolymers, such as styrene-olefin-styrene block copolymers or ethylene-propylene block copolymers. Such polymers may include hydrogenated grade styrol/ethylene-butylene/styrol (SEBS), styrene/isoprene/styrene (SIS), and styrol/ethylene-propylene/styrol (SEPS). The polymers may be nitrogen containing polymers. The polymers may be acrylics, sulphonated polymers, such as acrylamide sulphonated polymers, vinyl alcohols, vinyl pyrrolidone, polyethylene oxide and mixtures thereof.

In certain exemplary embodiments of the feminine undergarments disclosed here, all or a portion of the adhesive pre-applied to the body side of the strapless, coreless web comprises one or a combination of plasticizers compatible with the other components of the adhesive formulation. Suitable plasticizers include various commercially available plasticizers and others whose use will be readily apparent to those of ordinary skill in the art given the benefit of this disclosure. The plasticizer used in the adhesive employed on certain versions or embodiments of the undergarments disclosed here may be a combination of plasticizers, including, e.g., plasticizers that are liquid at room temperature. The plasticizer may be hydrophilic or hydrophobic. Exemplary suitable plasticizers include water, alcohols, polyhydric alcohols such as glycerol and sorbitol, and glycols and ether glycols such as mono- or diethers of polyalkylene glycol, mono- or diester polyalkylene glycols, polyethylene glycols (typically up to a molecular weight of about 600), glycolates, glycerol, sorbitan esters, esters of citric and tartaric acid, imidazoline derived amphoteric surfactants, lactams, amides, polyamides, quaternary ammonium compounds, esters such phthalates, adipates, stearates, palmitates, sebacates, or myristates, and combinations thereof. Particularly preferred are polyhydric alcohols, polyethylene glycol (with a molecular weight up to about 600), glycerol, sorbitol, water and mixtures thereof.

In certain exemplary embodiments of the feminine undergarments disclosed here, all or a portion of the adhesive pre-applied to the body side of the strapless, coreless web comprises one or a combination of micellising agents, such as a micellising polymer, compatible with the other components of the adhesive formulation. Suitable micellising agents include various commercially available micellising agents and others whose use will be readily apparent to those of ordinary skill in the art given the benefit of this disclosure. The micellising agent(s) used in the adhesive employed on certain versions or embodiments of the undergarments disclosed here may be, e.g., a lipid-micellising polymer or hypercoiling polymer that is understood to function to micellise and remove or isolate the grease and the like from the adhesive-skin interface. Without wishing to be bound by theory, such hypercoiling polymers are presently understood to aid in solvating surfactant micelles that contact hydrophobic skin contaminants, such as skin lipid or skin cream. Exemplary suitable hypercoiling polymers include, either alone or in combination: poly(maleic acid styrene), poly(maleic acid butyl vinyl ether), poly(maleic acid propyl vinyl ether), poly(maleic acid ethyl vinyl ether) and poly(acrylic acid ethyl acrylate). One example is an alternating copolymer of styrene and maleic anhydride. The adhesive can provide a biphasic structure, hydrophilic and hydrophobic.

In certain exemplary embodiments of the feminine undergarments disclosed here, all or a portion of the adhesive pre-applied to the body side of the strapless, coreless web comprises one or a combination of surfactants compatible with the other components of the adhesive formulation. Suitable surfactants include various commercially available surfactants and others whose use will be readily apparent to those of ordinary skill in the art given the benefit of this disclosure. The surfactant(s) used in the adhesive employed on certain versions or embodiments of the undergarments disclosed here may be, e.g., nonionic, cationic, anionic or amphoteric surfactants or compatible mixture thereof. Suitable nonreactive nonionic surfactants include, but are not limited to, the condensation products of an aliphatic alcohol, such as a fatty alcohol containing about 8 to about 20 carbon atoms in a straight or branched chain configuration, condensed with ethylene oxide. Examples of such nonionic ethoxylated fatty alcohol surfactants include at least certain members of the Tergitol™ 15-S series of nonylphenol ethoxylate (NPE) solvents sold by Union Carbide and Brij™ surfactants from ICI. Examples include Tergitol™ 15-S surfactants which are C₁₁-C₁₅ secondary alcohol polyethyleneglycol ethers, Brij™ 58 surfactant which is polyoxyethylene(20)cetyl ether and Brij™ 76 surfactant which is polyoxyethylene (10) stearyl ether. Other suitable nonreactive nonionic surfactants include, but are not limited to, those selected from the group consisting of the polyethylene oxide condensates of alkyl phenol containing from about 6 to 12 carbon atoms in a straight or branched chain configuration, with ethylene oxide. Examples of such nonionic surfactants are at least certain members of the Igepal™ CO and CA series of surfactants from Rhone-Poulenc. Igepal™ CO surfactants include nonylphenoxy poly(ethyleneoxy)ethanols and Igepal™ CA surfactants include octylphenoxy poly(ethyloneoxy)ethanols.

Other surfactants suitable for at least certain exemplary embodiments of the feminine undergarments disclosed here include, but are not limited to, those selected from the group consisting of block copolymers of ethylene oxide and propylene oxide or butylene oxide. Examples of such nonionic block copolymer surfactants include at least certain members of the Pluronic™ and Tetronic™ series of surfactants from BASF. Tetronic™ and Pluronic™ surfactants include ethylene oxide-propylene oxide block copolymers. Suitable examples include Pluronic™ L64, Pluronic™ L68 (commercially available from BASF Plc), Tetronic™ 1107 and Tetronic™ 1307 surfactants, among others. Other satisfactory nonreactive nonionic surfactants include, but are not limited to, at least certain members of the sorbitan fatty acid esters surfactants, polyoxyethylene sorbitan fatty acid ester surfactants and polyoxyethylene stearate surfactants. Examples of such fatty acid ester nonionic surfactants include at least certain members of the Span™, Tween™, and Myrj™ surfactants from ICI. Suitable Span™ surfactants include, e.g., at least certain C₁₂-C₁₈ sorbitan monoesters. Suitable Tween™ surfactants include, e.g., at least certain poly(ethylene oxide) C₁₂-C₁₈ sorbitan monoesters. Suitable Myrj™ surfactants include, e.g., at least certain poly(ethylene oxide) stearates.

At least certain suitable anionic surfactants include a hydrophobic moiety selected, e.g., from C₆ to C₂₀ alkyl, alkylaryl or alkenyl groups, and an anionic group selected, e.g., from sulfate, sulfonate, phosphate, polyoxyethylene sulfate, polyoxyethylene sulfonate, polyoxyethylene phosphate and the alkali metal salts, ammonium salts, and tertiary amino salts of such anionic groups. Anionic surfactants suitable for at least certain embodiments of the feminine undergarments disclosed here include, but are not limited to, C₆ to C₂₀ alkyl or alkylaryl sulfates or sulfonates, such as, e.g., sodium lauryl sulfate (commercially available as Polystep™ B-3 from Stepan Co.) and sodium dodecyl benzene sulfonate (commercially available as Siponate™ DS-10 from Rhone-Poulenc); polyoxyethylene (about C₆ to C₂₀) alkyl or alkylphenol ether sulfates with not more than about 20 ethylene oxide repeating units in the surfactant, e.g., about 15 units, such as Polystep™ B-1 (commercially available from Stepan Co.) and Alipal™ EP110 and 115 (commercially available from Rhone-Poulenc); (about C₆ to C₂₀) alkyl or alkylphenoxy poly(ethyleneoxy)ethyl mono-esters and di-esters of phosphoric acid and its salts, with not more than about 20 ethylene oxide repeating units in the surfactant, e.g., fewer than about 15 units, such as Gafac™ RE-510 and Gafac™ RE-610 (commercially available from GAF).

Other surfactants suitable for at least certain exemplary embodiments of the feminine undergarments disclosed here are cationic surfactants, including, but not limited to, at least certain cationic surfactants comprising quaternary ammonium salts in which at least one higher molecular weight group and two or three lower molecular weight groups are linked to a common nitrogen atom to produce a cation, and wherein the electrically-balancing anion is, e.g., a halide (bromide, chloride, etc.), acetate, nitrite, lower alkosulfate (methosulfate etc.) or the like. The higher molecular weight substituent(s) on the nitrogen may be, e.g., higher alkyl group(s) containing about 10 to about 20 carbon atoms, and the lower molecular weight substituents may be, e.g., lower alkyl of about 1 to about 4 carbon atoms, such as, e.g., methyl or ethyl, which may in certain exemplary embodiments be substituted, as with hydroxy. One or more of the substituents may include an aryl moiety or may be replaced by an aryl, such as benzyl or phenyl.

Without wishing to be bound by theory, it is presently understood that the surfactant may act to remove or isolate grease from the skin, forming pockets within the adhesive without substantially negatively impacting adhesion. Other common additives known in the art such as preservatives, antioxidants, pigments, mineral fillers and mixtures thereof may also be employed in the adhesive used in at least certain exemplary embodiments of the feminine undergarments disclosed here.

The body-side adhesive (to hold the undergarment to the wearer) may be pre-applied to the web of the feminine undergarments disclosed here in any amount and configuration adequate to provide suitable adhesion for its intended use. In certain exemplary embodiments, the pre-applied adhesive forms a continuous adhesive zone on the periphery of the body side of the strapless, coreless web. In certain exemplary embodiments the pre-applied adhesive is positioned intermittently about the periphery of the body side of the strapless, coreless web. In certain exemplary embodiments the pre-applied adhesive covers the entire body side of the strapless, coreless web. In certain exemplary embodiments the pre-applied adhesive covers at least a portion of a laterally extending anterior edge of the strapless, coreless web. In certain exemplary embodiments the pre-applied adhesive covers at least a portion of a laterally extending posterior edge of the strapless, coreless web. In certain exemplary embodiments the pre-applied adhesive covers at least the entire laterally extending anterior edge of the strapless, coreless web. In certain exemplary embodiments the pre-applied adhesive may cover at least a portion or the entire longitudinal sides extending from the anterior edge to the posterior edge of the strapless, coreless web. In certain exemplary embodiments the pre-applied adhesive covers at least the entire laterally extending posterior edge of the strapless, coreless web. In certain exemplary embodiments the pre-applied adhesive is all-natural, organic, or both. Such adhesive may be used with a web which is likewise all natural, organic, or both. The adhesive may be applied by any suitable method, including, e.g., by screen printing, brushing, extrusion, spray, etc.

In certain exemplary embodiments the pre-applied adhesive may be combined with grooves, ditches, ridges, imprints, dimples, crevices, a combination of these, and/or any other combination to provide optimal adherence to the wearer. In certain exemplary embodiments the adhesive nature of the undergarment may be due to grooves, ditches, imprints, dimples, crevices, a combination of these, and/or any other combination. One such example is U.S. Pat. No. 5,486,158. U.S. Pat. No. 5,486,158 describes a dressing having a moisture-absorbing adhesive sheet to be adhered to the skin and mucous membranes, with a layer of water swellable colloid which is dispersed in or mixed with a water insoluble viscous elastomer binder, wherein the adhesive sheet is provided with one or several grooves or ditches that fully or partly surround a central part of the adhesive sheet. In certain exemplary embodiments of the feminine undergarments disclosed here, the web is held in position on the wearer entirely or in part by suction. Such suction can be provided in one or more localized areas of the web by suitable material (in some cases referred to here as suction material) appropriately configured, e.g., by a rubbery material or the like formed as a round or other shape suction cup. In certain such embodiments, adhesive material is used to provide attachment to the wearer in addition to suction. In such embodiments the suction material, e.g., flexible or conformable silicone material, with or without pressure sensitive adhesive or other adhesive, is appropriately configured (e.g., in the general form of a suction cup) in one or more areas, preferably areas corresponding to smooth and generally hairless areas of the wearer's skin. Alternatively or in addition, suction can be provided in certain exemplary embodiments by pre-configuring the web of suction material (in some cases referred to here as a “suction web”) having an overall concave shape (toward the torso). In certain exemplary embodiments, such suction material forming the web will be a non-breathable material having shape memory. Optionally, suction material and/or adhesive is applied to the suction web, e.g., in a continuous perimeter on the web (i.e., forming a closed circle or other closed shape). Partially collapsing the concavity while applying such undergarment to the body will result in at least temporary suction to the wearer, comparable generally to a suction cup. Localized and/or overall suction can be provided by a combination of adhesive and ridges or other surface configuration(s) or the like in the body-side surface of the web, or any other suitable technique.

In certain exemplary embodiments employing suction material and having a removable and replaceable web portion, as disclosed above, some or all of the suction material optionally are located on the reusable part of the web. Certain exemplary embodiments having a suction web may have a removable and replaceable web portion. For example, a web may have an outer peripheral band of adhesive located entirely on an outer peripheral area of the body-side surface of the web. Such band of adhesive may provide localized and/or overall suction. The replaceable web portion may be located entirely within such outer peripheral area. In some such embodiments, a suction-providing adhesive band remains with the reusable part of the web and the replaceable part may or may not carry additional adhesive.

The web in at least certain exemplary embodiments of the undergarments disclosed here has good conformability to non-flat surfaces. The size and configuration are suitable for a selected portion of wearers, e.g., adult females, preadolescents, etc., as discussed above. In certain exemplary embodiments the strapless, coreless web is sized and configured to extend from its anterior end positioned on the female wearer between the navel and the mons veneris to its anterior end positioned at the perineum. In certain exemplary embodiments of the feminine undergarments, the strapless, coreless web is sized and configured to extend from the anterior end positioned on the female wearer between the navel and the mons veneris to the posterior end covering at least a portion of the buttocks. In certain exemplary embodiments of the feminine undergarments, the strapless, coreless web is sized and configured to extend from the anterior end positioned on the female wearer at the mons veneris to the posterior end positioned at the perineum. In certain exemplary embodiments of the feminine undergarments, the strapless, coreless web is sized and configured to extend from the anterior end positioned on the female wearer at the mons veneris to the posterior end covering at least a portion of the buttocks. Reference here to embodiments wherein the anterior end of the coreless, strapless web of the undergarments disclosed here being positioned on the female wearer between the navel and the mons pubis or mons veneris should be understood to mean that the anterior periphery or front edge of the web is at or below the navel but not posterior of the mons pubis. In certain exemplary embodiments, for example, it may be at the navel. In other exemplary embodiments it may be below the navel but higher (i.e., toward the navel) than the pubic hair region, so as to avoid or reduce contact of adhesive to pubic hair of the wearer. In other exemplary embodiments the anterior periphery of the web is between the top and the bottom of the mons pubis. In yet other exemplary embodiments it is at the top or the bottom of the mons pubis. Reference here to the anterior end of the coreless, strapless web of the undergarments disclosed here being positioned on the female wearer at the mons veneris means that the anterior periphery or front edge of the web is at the top or at the bottom of the mons pubis or is positioned between the top and the bottom of the mons pubis. Reference here to the posterior end of the coreless, strapless web of the undergarments disclosed here being positioned on the female wearer at the perineum means that the back edge of the web is adjacent to or on the perineum.

Certain exemplary embodiments of the feminine undergarments disclosed here are sized and configured to cover just the vulva. Certain exemplary embodiments are sized and configured to cover just the labia and vaginal opening, optionally including the glans clitoris and all or part of the clitoral hood. Certain exemplary embodiments are sized and configured to cover just the labia minora and vaginal opening, optionally including the glans clitoris and all or part of the clitoral hood. Certain exemplary embodiments are sized and configured to cover just the vaginal opening, optionally including the glans clitoris and all or part of the clitoral hood. Certain exemplary embodiments of the feminine undergarments disclosed here are sized and configured to extend rearwardly from just in front of the vulva. Certain exemplary embodiments of the feminine undergarments disclosed here are sized and configured to extend forward from just behind the vulva. In general, it should be understood that any given embodiment of the undergarments disclosed here may be sized and configured (i.e., shaped to sit properly over the vulva and any other covered tissue area(s) of the wearer) to have any combination of forward or anterior position (e.g., at the navel, just below the navel, mid-abdomen, above the mons veneris, mid-mons veneris, below or rearward of the mons veneris, rearward of the clitoral hood, at the forward edge of the vulva, etc.) and rearward or posterior version (e.g., at the rearward edge of the vulva, over some or all of the perineum, rearward of the perineum, over some or all of the buttocks, in the crease between the buttocks, optionally in contact with the anus, etc.).

In certain exemplary embodiments of the feminine undergarments disclosed here, the strapless, coreless web comprises a layer of one or a combination of woven, unwoven, knitted, lace, quilted stitched, or other fabric or other suitable material. The material may be breathable or non-breathable. For example, breathable materials may be breathable due exclusively to perforations or to the nature of the material itself, e.g., woven fabric, knitted fabric, breathable non-woven fabric, etc. The material may be flexible or form retaining. In duo-layer webs, the two layers each may be formed of any such materials mentioned here and may be formed of the same or different materials. In certain exemplary embodiments all or at least a portion of the strapless, coreless web of the feminine undergarment is sufficiently elastically stretchable to take on, in use, a concave shape (toward the wearer) to cup the wearer's vulva. In certain exemplary embodiments of the feminine undergarments disclosed here, all or at least a portion of the strapless, coreless web is seamless. It may be seamless and pre-configured or seamless and not pre-configured. As used here, a web that is entirely seamless may or may not have stitching, hemming, bonding or other treatment along some or all of the periphery of the web. For example, a seamless duo-layer web may have stitching, hemming, bonding or other treatment along some or all of the periphery of either or both layers to join the body-side layer to the outside layer. In certain exemplary embodiments the inner layer is attached continuously at its periphery to the outer layer, and it may be attached only at its periphery to the outer layer or also inside the periphery, e.g., even over the entire area of overlap between the layers. In certain exemplary embodiments the inner layer is attached. The inner layer may be attached by stitching, adhesively and/or other suitable techniques.

Certain embodiments of the feminine undergarments disclosed here are bio-occlusive, either fully bio-occlusive or semi-bio-occlusive, that is, they prevent or substantially prevent or reduce odor from the vulva or crotch area. In certain exemplary embodiments the strapless, coreless web comprises a non-permeable outer layer. In a mono-layer web version of such embodiments, the one layer of the web can be non-permeable. In duo-layer and tri-layer versions of such embodiments, one or more layers can be non-permeable. For example, one or both layers of a duo-layer web can be formed of a non-permeable material, e.g., a permeable fabric treated to make it non-permeable or a naturally non-permeable material, such as plastic film or the like. Alternatively, the body-side layer of a duo-layer web may be absorbent, e.g., cotton fabric, while the outer layer is non-permeable. Likewise, one, two or all layers of a tri-layer web can be formed of a non-permeable material. In such embodiments the term “non-permeable outer layer” refers to a layer that does not wick moisture and does not allow substantial diffusion of odor through the layer. Such non-permeable layer can be an outer layer in the sense that it is not a body side layer, i.e., it is not the layer of the web that lies closest to the vulva of the wearer. In certain exemplary embodiments comprising a non-permeable layer, the adhesive extends uninterrupted around the entire perimeter or substantially the full perimeter of the web, optionally covering the entire body side surface of the web, such that diffusion of odor from the vulva can be prevented or substantially reduced cooperatively by the adhesive and the non-permeable layer. In certain exemplary embodiments the non-permeable layer is sized to the full dimensions of the other layer(s) of the web. That is, the non-permeable layer in such embodiments fully covers the one or more inner layer(s) of the web. Suitable materials for the non-permeable layer will be apparent to those of ordinary skill in the art given the benefit of this disclosure, including numerous commercially available materials. Materials suitable for at least certain exemplary embodiments of the undergarments disclosed here that comprise a non-permeable layer include, for example, thin layers of polyvinyl chloride film or other vinyl material, polyethylene film, polyurethane film, silicone film, or any other suitable substance or material. Additional suitable materials will be readily apparent to those of ordinary skill in the art given the benefit of this disclosure.

Certain exemplary embodiments of the feminine undergarments disclosed here optionally comprise one or more active agents, e.g., a perfume, cologne, essential oils, flavorant, pharmaceutical, nutriceutical, herbaceutical, nutrient, phytonutrient, probiotic, anti-viral, viricide, antibacterial, antiperspirant, anti-fungal, antiseptic, deodorant, odor neutralizer or suppressant, anti-microbial, microbicide, anti-itching agent, desiccant powder or other drying agent, cooling agent, vitamins, antioxidant, skin rejuvenating agent, pH stabilizing agent, libido enhancement and/or stimulant, hormonal balancing, sun protectant, lubrication and any other suitable agent(s) medically or cosmetically useful, or a mixture of any two or more of them. Exemplary antioxidants include gallic acid, Vitamin C, ascorbic acid, Vitamin E. Those of ordinary skill in the art will understand that there is overlap amongst the categories of active agents listed above. The optional active agent(s) may be provided in or on the web and/or the adhesive and/or any other component of the undergarment, including, optionally, for example, a replaceable component, as discussed above. In certain exemplary embodiments the anti-itching agent comprises starch, e.g., corn starch, kaolin, zinc oxide, such as Mexsana (an antiseptic medicated powder made predominantly from corn starch), Calamine (a mixture of zinc oxide (ZnO) with about 0.5% iron(III) oxide (Fe₂O₃)), etc. In certain exemplary embodiments the active agent comprises an odor neutralizing agent selected from zinc and zinc compounds, silver and silver compounds, activated carbon, baking soda, etc., or a combination of any of them.

Certain exemplary embodiments of the feminine undergarments disclosed here are odor neutralizing and/or odor suppressing agents. Such embodiments optionally are also odor impermeable as described above. Odor neutralizing and/or suppressing embodiments may contain one or a combination of odor neutralizing agents, e.g., deodorant, anti-perspirant, antibacterial agent, antifungal agent, odor absorbent, and/or other odor neutralizer. For example, suitable odor neutralizing and/or suppressing agent may be provided in or on the web of the undergarment. Numerous odor neutralizing agents and/or suppressing agents suitable for use in the undergarments disclosed here will be apparent to those skilled in the art given the advantage of this disclosure. Exemplary neutralizing agents and/or suppressing agents suitable for at least certain embodiments of the undergarments include activated charcoal, zinc oxide, sodium bicarbonate, fragrance, aluminum zirconium tetrachlorohydrex gly and/or other aluminum chlorohydrates, 5-chloro-2-(2,4-dichlorophenoxy)phenol) or other wide spectrum antibacterial and antifungal agent, etc. In certain exemplary embodiments the neutralizing agent and/or suppressing agent is time-released, that is, it is slowly released over time to the wearer in a manner the same as or similar to drug delivery via well known transdermal patch techniques, as the undergarment is being worn. At least certain such embodiments can advantageously provide continual freshness for the wearer.

Certain exemplary embodiments of the feminine undergarments disclosed here are perfume and/or cologne agents including but not limited to natural and/or synthetic scents and/or oils, and/or any other suitable agent(s) medically or cosmetically useful, or a mixture of any two or more of them.

Certain exemplary embodiments of the feminine undergarments disclosed here are essential oils including but not limited to lavender, peppermint, eucalyptus, rose oil, and/or any other suitable agent(s) medically or cosmetically useful, or a mixture of any two or more of them.

Certain exemplary embodiments of the feminine undergarments disclosed here are flavorants including but not limited to vanilla, peppermint, mint, citrus, melon, berry, and/or any other suitable agent(s) medically or cosmetically useful, or a mixture of any two or more of them.

Certain exemplary embodiments of the feminine undergarments disclosed here are pharmaceutical agents including but not limited to prescription and/or non prescription agents and/or any other suitable agent(s) medically or cosmetically useful, or a mixture of any two or more of them.

Certain exemplary embodiments of the feminine undergarments disclosed here are nutraceutical, phytonutirents, nutrient agents, and/or phystochemic agents include but not limited to resveratrol, sulforaphane, anthocyanins, gallic acid, malvidin, naringenin, probiotics, and/or any other suitable agent(s) medically or cosmetically useful, or a mixture of any two or more of them.

Certain exemplary embodiments of the feminine undergarments disclosed here are herbaceutical agents including but not limited to aloe vera, agaricus blazei, blackberry (Rubus fruticosus), black raspberry (Rubus occidentalis), calendula (Calendula officinalis,) German chamomile (Matricaria chamomilla), green tea (Camelia sinensis), Hibiscus sabdariffa, nigella saliva (Black cumin), oregano (Origanum vulgare), peppermint oil, phytolacca or pokeweed, pomegranate, soy and other plants that contain phytoestrogens (plant molecules with estrogen activity), black cohosh, and/or any other suitable agent(s) medically or cosmetically useful, or a mixture of any two or more of them.

Certain exemplary embodiments of the feminine undergarments disclosed here are probiotic agents including but not limited to lactic acid bacteria and bifidobacteria, and/or any other suitable agent(s) medically or cosmetically useful, or a mixture of any two or more of them.

Certain exemplary embodiments of the feminine undergarments disclosed here are antiperspirant agents including but not limited to aluminium chloride, aluminium chlorohydrate, baking soda, aluminium-zirconium, aluminium zirconium trichlorohydrex gly, and/or any other suitable agent(s) medically or cosmetically useful, or a mixture of any two or more of them.

Certain exemplary embodiments of the feminine undergarments disclosed here are deodorant agents including but not limited to fragrance (parfum), talc, glycerin, (sunflower) seed oil, baking soda, and/or any other suitable agent(s) medically or cosmetically useful, or a mixture of any two or more of them.

Certain exemplary embodiments of the feminine undergarments disclosed here are cooling agents including but not limited to menthol, mint, Asensa DS 826, and/or any other suitable agent(s) medically or cosmetically useful, or a mixture of any two or more of them.

Certain exemplary embodiments of the feminine undergarments disclosed here are vitamins including but not limited to vitamin C, vitamin E, vitamin A (beta-carotene), vitamin B12, vitamin B6, and/or any other suitable agent(s) medically or cosmetically useful, or a mixture of any two or more of them.

Certain exemplary embodiments of the feminine undergarments disclosed here are skin rejuvenating agents including but not limited to emu oil, squalene oil, collagen, alpha lipoic acid, green tea extract, grape seed extract, arnica, ginseng, ascorbic acid, ginkgo biloba, allantoin, centella asiatic extract, fruit acids, hyaluronic acid, retinyl palmitate, tetrahexyldecyl ascorbate, beta-carotene, vitamin E, flower extract(s), seaweed, algae extract, and/or any other suitable agent(s) medically or cosmetically useful, or a mixture of any two or more of them.

Certain exemplary embodiments of the feminine undergarments disclosed here are pH stabilizing agents including but not limited to sodium hydroxide, methylparaben sodium, sodium bicarbonate, and/or any other suitable agent(s) medically or cosmetically useful, or a mixture of any two or more of them.

Certain exemplary embodiments of the feminine undergarments disclosed here are libido enhancement, libido arousal and/or stimulant agents including but not limited to herbals, testosterone, and/or any other suitable agent(s) medically or cosmetically useful, or a mixture of any two or more of them.

Certain exemplary embodiments of the feminine undergarments disclosed here are hormonal balancing agents including but not limited to estradiol, progesterone, testosterone, and/or any other suitable agent(s) medically or cosmetically useful, or a mixture of any two or more of them.

Certain exemplary embodiments of the feminine undergarments disclosed here are sun protectant agents. Such agents may filter, block, reflect, absorb, scatter, and/or other suitable means for protecting the wearer from the sun's rays including ultraviolet (UV) radiation. Some exemplary examples include but are not limited to polypropylene, natural or synthetic indigo dyed material, canvas, twill weaves, and/or other suitable agent(s) medically or cosmetically useful, or a mixture of any two or more of them.

Certain exemplary embodiments of the feminine undergarments disclosed here are lubricating agents including but not limited to essential oils, pogostemon cablin, olea europaea, tocopherol, cinnamomum cassia, salvia sclarea, canaga odorata, cogostemon cablin, glycerin, petrolatum, and/or other suitable agent(s) medically or cosmetically useful, or a mixture of any two or more of them.

Certain exemplary embodiments of the feminine undergarments disclosed here are pharmaceutical agents which may be prescription and/or non prescription drugs including but not limited to natural, synthetic, semi-synthetic, and/or other suitable agent(s) medically or cosmetically useful, or a mixture of any two or more of them.

In certain exemplary embodiments the active agent comprises one or more antibacterial agents, antiseptics, microbicides, and/or antivirals, which optionally may be selected, for example, from alcohols (e.g., ethanol (60-90%), 1-propanol (60-70%), 2-propanol/isopropanol (70-80%) and mixtures of any of them), tincture of iodine, certain cationic surfactants (e.g., (benzalkonium chloride 0.05-0.5%, chlorhexidine 0.2-4.0%, octenidine dihydrochloride 0.1-2.0%, etc. and mixtures of any of them), quaternary ammonium compounds (or Quats or QACs) (e.g., benzalkonium chloride (BAC), cetyl trimethylammonium bromide, cetylpyridinium chloride, benzethonium chloride and mixtures of any of them), boric acid, hydrogen peroxide, organomercury antiseptics (e.g., mercurochrome, bis-(phenylmercuric) monohydrogenborate and mixtures of any of them), octenidine dihydrochloride and bis-(dihydropyridinyl)-decane derivative (e.g., 0.1-2.0% concentration), phenol, thymol, triclosan, sodium 3,5-dibromo-4-hydroxybenzenesulfonate, sodium chloride, sodium or calcium hypochlorite, sodium bicarbonate (NaHCO₃), and mixtures of any of them.

In certain exemplary embodiments the active agent comprises an antibacterial agent selected from pharmaceutical prescription medication and/or over-the counter product with either specific coverage or broad coverage of gram positive, gram negative, and/or anaerobic organism(s). Some agents include but are not limited to bacitracin, polymyxin B, and neomycin. Some medication classes include, cephalosprins, polymixins, quinolones, sulfonamides, aminoglycosides, macrolides tetracyclines, cyclic lipopeptides, glycylcylides, and oxazolidinones. In certain exemplary embodiments the active agent comprises an antimicrobial agent selected from zinc, silver, citric acid, tea tree oil, lavender oil, mint oil, lemon oil, and pharmaceutical agents. In certain exemplary embodiments the active agent comprises an antifungal agent selected from selenium, tea tree oil, terconazole, miconazole, clotrimazole, tioconazole, and boric acid.

Certain exemplary embodiments of the feminine undergarments disclosed here comprise a transdermal system, that is, a system for delivery to the wearer of one or more pharmaceutical, nutriceutical, nutrient, probiotic, anti-viral, viricide, antibacterial, anti-fungal, anti-microbial, microbicide, hormonal, and/or any other applicable agent listed above and/or any applicable agent medically or cosmetically useful and suitable, or a mixture of any two or more of them. Certain such embodiments can deliver an agent into the skin of the wearer of the undergarment in a time release manner. For example, the agent may be presented in a matrix positioned to contact the wearer's skin. Such matrix can be the web or a portion of the web, e.g., a layer of the web, or some or all of the adhesive carried by the web to hold the undergarment in place, or it can be a different material carried by the web or another portion of the undergarment. Certain suitable transdermal systems include but are not limited to 3M Solid Microstructured Transdermal System Microneedle Delivery, Technologies3M Drug In Adhesive Transdermal Delivery Passive Transdermal Technology, Durect Corporation Transdur™ Transdermal technology and/or any other suitable transdermal application.

In certain exemplary embodiments the active agent comprises an odor neutralizing or other active agent comprising zinc, silver, activated carbon, baking soda or the like, or a mixture of any of them. In certain exemplary embodiments the active agent comprises an antibacterial agent selected from pharmaceutical prescription medication and/or over-the counter product with either specific coverage or broad coverage of gram positive, gram negative, and/or anaerobic organism(s).

Certain exemplary embodiments of the feminine undergarments disclosed here are odor neutralizing. Such embodiments optionally are also odor impermeable as described above. Odor neutralizing embodiments may contain one or a combination of odor neutralizing agents, e.g., deodorant, anti-perspirant, antibacterial agent, antifungal agent, odor absorbent, and/or other odor neutralizer. For example, suitable odor neutralizing agents may be provided in or on the web of the undergarment. Numerous odor neutralizing agents suitable for use in the undergarments disclosed here will be apparent to those skilled in the art given the advantage of this disclosure. Exemplary neutralizing agents suitable for at least certain embodiments of the undergarments include activated charcoal, zinc oxide, sodium bicarbonate, fragrance, aluminum zirconium tetrachlorohydrex gly and/or other aluminum chlorohydrates, 5-chloro-2-(2,4-dichlorophenoxy)phenol) or other wide spectrum antibacterial and antifungal agent, etc. In certain exemplary embodiments the neutralizing agent is time-released, that is, it is slowly released over time to the wearer in a manner the same as or similar to drug delivery via well known transdermal patch techniques, as the undergarment is being worn. At least certain such embodiments can advantageously provide continual freshness for the wearer.

Certain exemplary embodiments of the undergarments comprise one or more longitudinal, lateral and/or angled extensions, e.g., one or more side, anterior, and/or posterior tabs, such as at least one right-side lateral extension, at least one left-side lateral extension between the anterior end and the posterior end, at least one tab at or near the anterior end, and/or at least one tab at or near the posterior end. Such one or more lateral, longitudinal, and/or angled extensions can carry all or at least a portion of the pre-applied adhesive. Lateral extensions can be sized and positioned to be adhered to any skin area adjacent to the web of the undergarment, e.g., to either or both thighs, to the perineum, to either or both sides of the buttocks, to the mons veneris or lower abdomen, etc. Certain exemplary embodiments have a single right-side adhesive tab and a single left side adhesive tab, sized and positioned to be adhered to the inside of the right and left thigh, respectively, of the wearer. Certain exemplary embodiments have one or more lateral extension for adhesion to the buttocks. Certain exemplary embodiments have one or more longitudinally extending adhesive tabs extending from the posterior edge of the web and/or from the anterior edge of the web. In certain exemplary embodiments adhesive tabs extend from the web at an angle between lateral and longitudinal, e.g., at an angle of from 30° to 60° from the front-to-back axis of the web. Such adhesive tabs can be used in embodiments with or without preconfigured webs. For example, certain embodiments of the feminine undergarments comprise a symmetrical pair of laterally extending tabs, each carrying adhesive positioned for contact with a corresponding one of the inner thighs of a wearer. The tabs may be unitary with the web, e.g., formed by the same sheet or film of fabric or other material forming the web. In multi-layer webs, such tabs may be formed of all or fewer than all of the web layers. Alternatively, the tabs may be formed of the same or different material and attached to the web, e.g., by stitching, Velcro®, adhesive, etc.

In certain exemplary embodiments of the feminine undergarments disclosed here, the entire strapless, coreless web or at least a portion of the posterior end of the web is pre-configured to be body side concave to follow contours of the buttocks. In certain exemplary embodiments of the feminine undergarments disclosed here, at least a portion of the anterior and/or posterior end of the strapless, coreless web is pre-configured to be body side concave to cup the female wearer's vulva. In certain exemplary embodiments of the feminine undergarments disclosed here, at least a portion of the anterior and/or posterior end of the strapless, coreless web is pre-configured to be overall body-side laterally convex, either with or without a laterally centered concavity to receive and cup the wearer's vulva. The overall convexity could, e.g., extend from a right-side edge of the web laterally to a left-side edge, where such side edges of the web are configured to be at the wearer's upper right and left thigh, respectively. The term “pre-configured” as used here with reference to the web of certain exemplary embodiments of the minimalist undergarments disclosed here, means that the web has a particular shape in the free state, e.g., as packaged for sale, rather than being simply flexible and shapeless (other than its perimeter configuration, e.g., oval, tear-drop, egg-shaped, tabbed, etc.) in the sense that the web is entirely flexible (e.g., as a typical piece of cotton cloth or the like). The shape may be, for example, flat or a simple curvature, such as body side concave (meaning all or a portion of the upper side of the web is concave toward the torso) or a more complex curvature, such as saddle shaped, i.e., having both concave and convex curvatures toward the torso, e.g., being overall concave toward the body or cup-shaped in the longitudinal direction (i.e., anterior-to-posterior) and overall convex toward the body in the lateral direction (i.e., from side-to-side or thigh-to-thigh). The pre-configured shape also may have localized concavities (again, meaning toward the torso), e.g., to accommodate anatomical features. In at least certain exemplary embodiments having a pre-configured web, the web has shape memory. That is, it has resiliency to assume or return wholly or at least substantially toward its pre-configuration in the free state, i.e., when not being held in a different shape such as by the adhesive when being worn. It should be understood that a pre-configured web optionally is still somewhat flexible, elastic and/or pliable. For example, reference here to certain exemplary embodiments wherein a portion of the posterior end of the strapless, coreless web is pre-configured to be body side concave to follow the contours of the user's buttocks (i.e., the user's lower backside formed primarily by fat and the gluteal muscles) means that at least that a posterior portion of the web in the free state, e.g., as first removed from its packaging, assumes one or a pair of body side concavities (i.e., concavities facing the body) shaped to follow generally the curvature of a wearer's buttocks. Similarly, reference here to a portion of the strapless, coreless web being pre-configured to cup the wearer's vulva means that at least a portion of the web between the anterior end and the posterior end is pre-configured to be body side concave in the free state (i.e., to be concave toward the wearer in use) in the longitudinal direction. In certain exemplary embodiments the undergarment is packaged in a substantially flat condition (being either symmetrical or asymmetrical), and upon being removed from its packaging assumes a different shape, i.e., its pre-configured shape, e.g., a shape accommodating a typical female vulva. The packaging is used in certain such embodiments to constrain the undergarment to a shape other than the pre-configured shape it assumes in the free state once removed from the packaging. In certain exemplary embodiments the undergarment is packaged to maintain its pre-configured shape.

As mentioned previously, certain exemplary embodiments provides at least some “fill-in” function of the wearer's creases, anus, vulva, and/or other natural or artificial areas. Some exemplary embodiments may form a temporary, full circumference fill-in of at least some of the vulva. Certain, but not all embodiments of the undergarments also provide fill-in contact with the anus and/or the skin surrounding the anus so as to form a temporary, full circumference fill-in at least over some of the anus. Some fill-in materials include, hydrocolloid, hydrocolloid gel, silicone, foam, silicone, algae, and/or any and/or any other suitable agent(s) medically or cosmetically useful, or a mixture of any two or more of them. In certain exemplary embodiments, the fill-in material may be in the form of a gel, solid, liquid, and/or any mixture of two or more of these. The fill-in material may be at least part of the main web, at least part of a secondary web, at least part of the adhesive, at least part of the entire undergarment, removable, replaceable, and/or a combination of one or more of these.

As mentioned previously, certain exemplary embodiments provide at least some molding contact with the vulva and/or the skin surrounding the vulva so as to form a temporary, full circumference mold over at least some of the vulva. Certain, but not all embodiments of the undergarments also provide molding contact with the anus and/or the skin surrounding the anus so as to form a temporary, full circumference mold over and/or in at least some of the anus. Some molding materials include hydrocolloid, hydrocolloid gel, silicone, foam, algae, and/or any and/or any other suitable agent(s) medically or cosmetically useful, or a mixture of any two or more of them. In certain exemplary embodiments, the molding material may be in the form of a gel, solid, liquid, and/or any mixture of two or more of these. The mold may be at least part of the main web, at least part of a secondary web, at least part of the adhesive, at least part of the entire undergarment, removable, replaceable, and/or a combination of one or more of these.

A pre-configured web may be formed by any suitable means. For example, the web may be thermoformed, pleated, seamed, etc. Pre-configuring can be achieved entirely or in part by treatment of all or a portion of the periphery of the web. In certain exemplary embodiments of the feminine undergarments disclosed here having a duo-layer web, the entire strapless, coreless web or at least a portion of the web is pre-configured at least in part by attachment of the inner layer to the outer layer. For example, a concave web shape can be achieved by ruching, or otherwise gathering web material at its periphery, either with or without additional shaping techniques. Optionally, pre-configuring the web is achieved entirely or in part by using different treatments for different portions of the periphery of the web. For example, the web material can be gathered more in one portion of the periphery than at another. Also, for example, edge gathering can be used in conjunction with thermoforming, pleating, seaming and/or other techniques to achieve the concave or other pre-configured web shape desired for a particular embodiment of the feminine undergarments disclosed here.

The web in certain exemplary embodiments of the feminine undergarments disclosed here is pre-configured to follow the contours of the vulva, with or without also being configured to follow the contours of the buttocks. Embodiments configured to follow the contours of the vulva may be generally with or without having other configurations, such as a saddle, concave and/or convex configuration.

It is typically desirable that at least the anterior and posterior edges of the web be smooth, i.e., have a low profile, i.e., have a thickness little or no greater than the thickness of the layer(s) of fabric used to form the web. For example, the periphery may be treated with merely a thin layer of adhesive, optionally being all or some of the same adhesive used for adherence to the wearer. The entire peripheral edge of the web is smooth in certain exemplary embodiments. In certain exemplary embodiments the entire web, including the periphery and the area of the web within the periphery, is smooth in order to best facilitate a smooth appearance for the wearer under any clothing type.

In accordance with another aspect of this disclosure, feminine undergarments in accordance with the principles disclosed and discussed above further comprise a removable protective layer for the adhesive. Such protective layer may be formed of one or more pieces or sheets of paper, plastic, etc., e.g., paper treated with silicone ant-stick material or the like to facilitate removal by the wearer prior to being worn. In certain exemplary embodiments the layer is formed as a sealed envelop enclosing the undergarment or at least the strapless, coreless web or portions thereof having pre-applied adhesive. For example, all or portions of the envelope or other protective layer can be formed of opaque, clear or translucent cellophane or other plastic film material or other material. In certain exemplary embodiments the protective layer has printed information, drawings illustrating its removal, drawings showing use of the undergarment, etc. In certain exemplary embodiments the protective layer is suitable to be reapplied to the adhesive on the web after removal.

In accordance with another aspect of this disclosure, a package of feminine undergarments is provided, comprising multiple feminine undergarments in accordance with the principles disclosed and discussed above, and outer packaging containing at least the multiple feminine undergarments. Optionally, the packaging also contains other undergarments, other clothing items, instructions for use of the feminine undergarments, printed materials, e.g., printed materials providing instructions for use of the undergarments, etc. Optionally the feminine undergarments contained in the package each also has a removable protective layer covering at least the adhesive and removable by the wearer prior to use of the undergarment. The packaging may be, for example, plastic, e.g., clear, translucent or opaque plastic, rigid or flexible plastic, etc., cardboard, paper, cellophane, or other suitable material(s) or any combination of two or more materials. Optionally the package is re-closable after first being opened by the consumer, to continue containing others of the multiple undergarments originally included in the package.

The undergarment optionally is decorated. For example, in certain exemplary embodiments the outside surface and/or the inside surface of the web is colored or shows a decorative pattern or other decorative image, e.g., a printed, silk screened, embedded, embossed, impressed, and/or quilted pattern. Any desired colors, images and patterns can be used, for example, geometric patterns, animal prints, etc. In certain exemplary embodiments the undergarment is skin colored, that is, it has the color of one or another demographic group. In addition, the undergarment optionally may have decorative features such as frills, ribbons, bows, appliqués, cut-outs and the like. Such features may be added to the web in any suitable manner, e.g., by stitching, adhesive, cut-outs, etc. In certain exemplary embodiments the undergarment has a decorative edge treatment along some of all of its peripheral edge. Such decorative edge treatment may or may not be functional as well as decorative.

It should be understood that the various features described above or illustrated in the appended drawings can be used together in any combination or permutation. All such combinations and permutations, whether or not specifically, individually mentioned expressly in this disclosure or shown in the drawings are within the scope of the inventive subject matter.

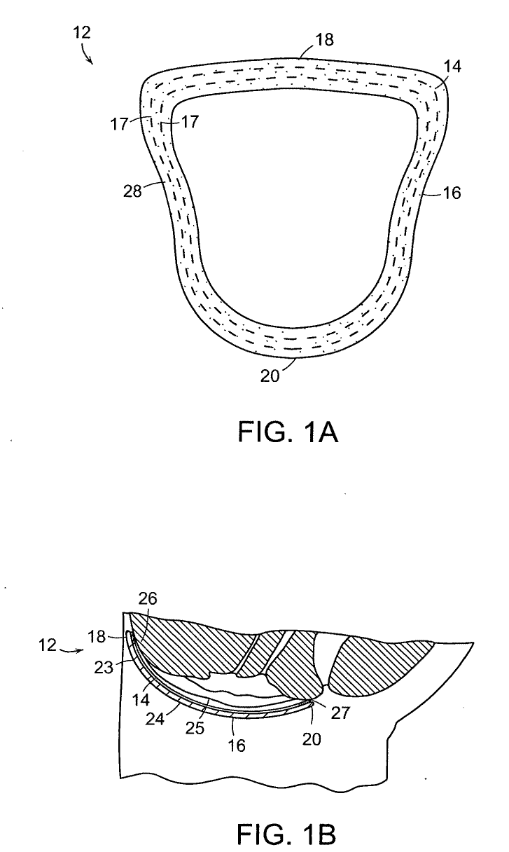

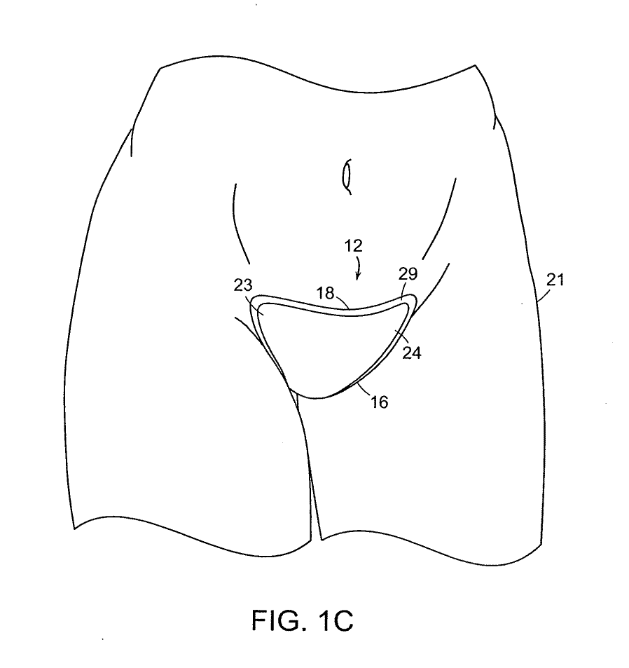

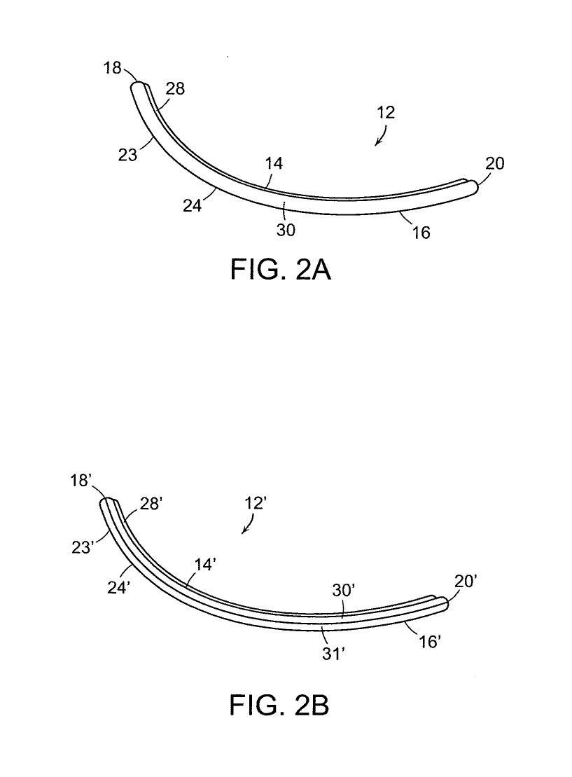

Referring now to the drawings, FIGS. 1A-1C and 2A show a non-limiting example of the feminine undergarments disclosed here. Specifically, FIG. 1A shows the upper or body side 14 of a feminine undergarment 12 comprising a strapless, coreless web 16 having an anterior end 18, a posterior end 20. In FIG. 1C showing the undergarment 12 in position on an adult female 21, an anterior portion 23 of the outside surface 24 of the web 16 of the undergarment 12 is seen. The web 16 is sized and configured between the anterior end 18 and the posterior end 20 to cover, in use, at least an adult female wearer's vulva 25 and, more particularly in this embodiment, as best seen in the cross-section view of FIG. 1B, from the wearer's mons pubis 26 to the perineum 27. Exemplary web sizes include (i) a longitudinal dimension from the anterior end 18 to the posterior end 20 of from about 5 cm to about 40 cm, or from about 5 cm to about 35 cm, e.g., from about 10 cm to about 15 cm or from about 8 cm to about 12 cm, and ii) lateral dimension from the left side to the right side at the longitudinal mid-point or center of the vulva of from about 3 cm to about 10 cm, or from about 3 cm to about 20 cm, e.g., from about 3 cm to about 5 cm or from about 3 cm to about 8 cm. For measurements that may also cover the buttocks (may extend from navel and/or vulva (anterior end) to posterior of anal opening. Exemplary web sizes include (i) a longitudinal dimension from the anterior end to the posterior end of from about 5 cm to about 65 cm, or from about 6 cm to about 68 cm, e.g., from about 17 cm to about 26 cm or from about 12 cm to about 30 cm.

The undergarment 12 has pre-applied adhesive 28 on the body side 14 of the web, suitable for prolonged contact to human skin and sufficiently adherent to removably hold the undergarment in place during use. It can be seen that the periphery of the web is smooth. The web 16 has guide 17 in the form of multiple, generally parallel lines of perforations. The perforation lines extend generally parallel to any part and/or all of the peripheral edge of the web, whereby a peripheral portion of the web may optionally be removed by hand tearing, cutting with scissors, etc. to reduce the size of the web. Alternatively, only a single line of perforations could be provided. In embodiments such as that of FIG. 1 having multiple lines, the number of such lines and the spacing between them will generally determine the size adjustments facilitated. It will be apparent to those of ordinary skill in the art that alternative guides (e.g., printed lines, etc.) could be provided either instead of or in addition to the perforation lines shown in the exemplary embodiment of FIG. 1.

FIG. 2A shows a side elevation view of the embodiment of FIGS. 1A-1C, specifically, a mono-layer web version. The mono-layer version has a single layer of material and/or combination of materials 30 forming the web 16. FIG. 2B shows a section view of an alternative version of the embodiment of FIG. 1, specifically, a duo-layer web version, wherein corresponding features are designated by corresponding reference numerals with a prime mark. The duo-layer version has two layers of material and/or combination of materials 30′ and 31′ forming the web 16′. The edge treatment 24′ in the embodiment of FIG. 2B serves in the embodiment of FIG. 2B to join the first layer of fabric 30′ to the second layer of fabric 31′.

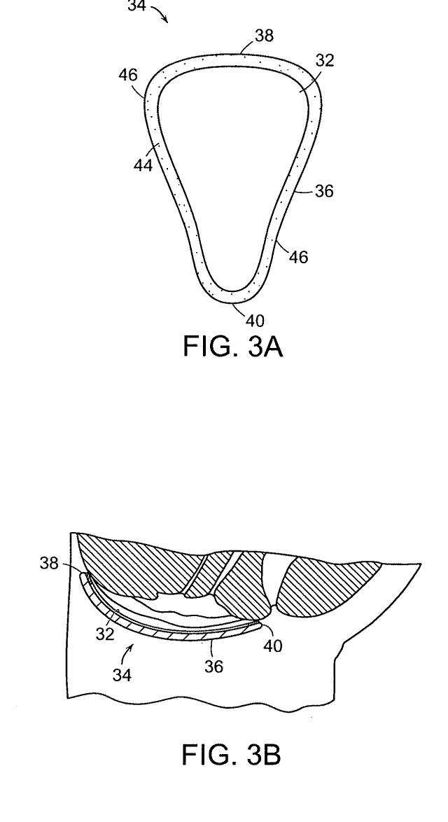

FIGS. 3A-3B show another embodiment of the feminine undergarments disclosed here, showing the body side 32 of undergarment 34 comprising a strapless, coreless web 36. Web 36 has an anterior end or edge 38, a posterior end or edge 40, and an outside (not seen). The web 36 is sized and configured to cover, in use, at least an adult female wearer's vulva. More particularly in this embodiment, the web is more narrow laterally than the embodiment of FIGS. 1A-1C and 2A, while still being sized to extend from the wearer's perineum to the mons pubis. The undergarment 34 has pre-applied adhesive 44 on the body side 32 of the web, suitable for prolonged contact to human skin and sufficiently adherent to removably hold the undergarment in place during use. It can be seen that the periphery 46 of the web 36 is smooth, with no edge treatment. Optionally, in this or multi-layered web embodiments, edge treatment comprising, for example, stitching, adhesive bonding, quilting, embossing, cut-outs, ribbons and/or other decorative or functional frills and/or other suitable treatment, can be included. Such edge treatment can serve in certain such embodiments as a joining feature for multiple web layers and/or to control fraying or otherwise improve durability or comfort.

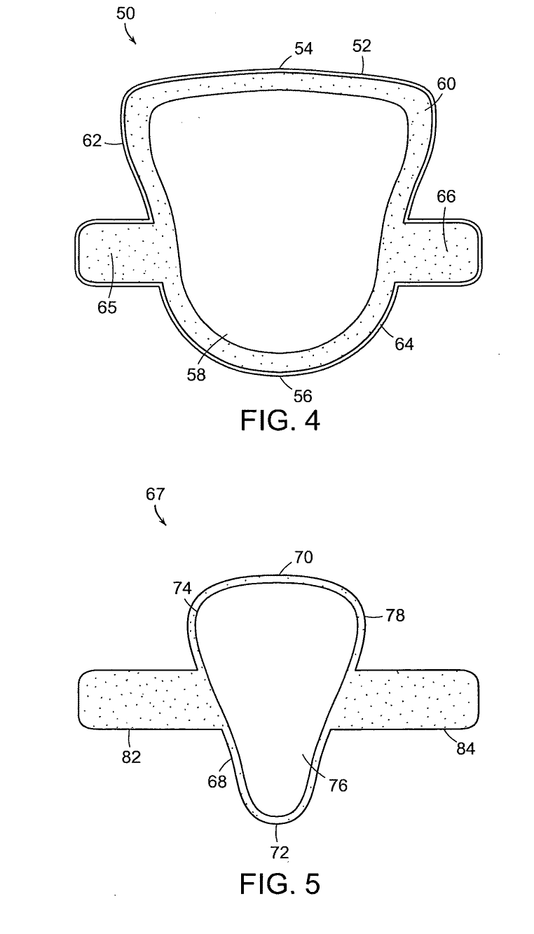

FIG. 4 shows another embodiment of the feminine undergarments disclosed here. Undergarment 50 has a strapless, coreless web 52 with anterior end or edge 54, posterior edge 56, body side 58 and outside (not seen). The web 52 is sized and configured to cover, in use, at least an adult female wearer's vulva, extending from the wearer's perineum to the mons pubis. The undergarment 50 has pre-applied adhesive 60 on the body side 58 of the web, suitable for prolonged contact to human skin and sufficiently adherent to removably hold the undergarment in place during use. The pre-applied adhesive 60 is present in a peripheral band and absent from the center area which would contact the vulva. It will be within the ability of those of ordinary skill in the art to select a particular band width or other configuration suitable to the particular intended use or application. It can be seen that the periphery 62 of the web 52 has edge treatment 64 which may optionally be decorative and/or, in a multi-layer embodiment, can serve to join the multiple layers of the web, etc. In addition, this embodiment has a right-side lateral extension or tab 65 and a left-side lateral extension or tab 66 between the anterior end 54 and the posterior end 56. The tabs are sized and positioned to be adhered to the right and left thigh, respectively, of the wearer. The tabs carry a portion of the pre-applied adhesive 60 and, therefore, aid in holding the undergarment to the wearer in proper position during use.

FIG. 5 shows another embodiment of the feminine undergarments disclosed here. Undergarment 67 has a strapless, coreless web 68 with anterior end or edge 70, posterior edge 72, body side 76 and outside (not seen). The web 68 is sized and configured to cover, in use, at least a female wearer's vulva, extending from the wearer's perineum to the navel. More particularly in this embodiment, the web is more narrow laterally than the embodiment of FIG. 4, but similar to the embodiment of FIG. 4 this embodiment has right-side and left-side lateral tabs between the anterior end 70 and the posterior end 72. The undergarment 67 has pre-applied adhesive 74 on the body side 76 of the web, suitable for prolonged contact to human skin and sufficiently adherent to removably hold the undergarment in place during use. The laterally extending tabs 82, 84 are sized and positioned to be adhered to the right and left thigh, respectively, of the wearer. The tabs carry a portion of the pre-applied adhesive 74 and, therefore, aid in holding the undergarment to the wearer in proper position during use.

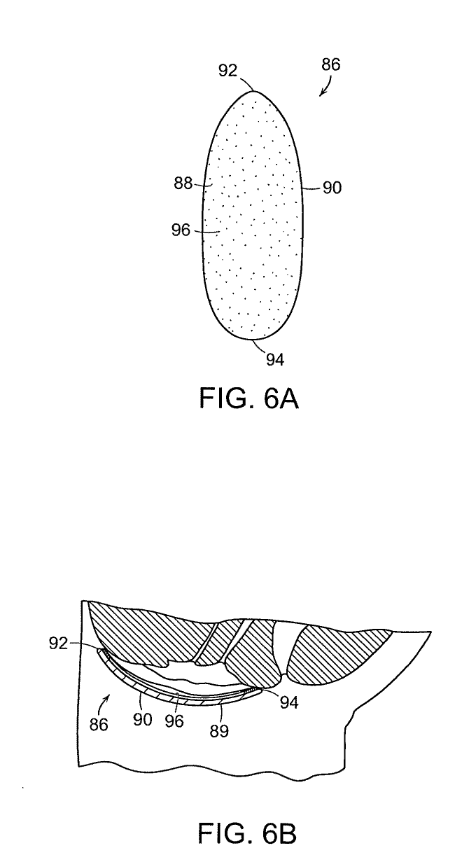

FIGS. 6A and 6B show another embodiment of the feminine undergarments disclosed here, specifically, an embodiment sized and configured to cover, in use, at least an adult female wearer's vulva, but extending only from the perineum to about the bottom of the mons pubis. Undergarment 86 comprises a strapless, coreless web 90 having anterior end 92, posterior end 94, body side 88 and an outside surface 89. The web of this embodiment is seen to be more narrow laterally than the embodiment of FIG. 1A. The undergarment 86 has pre-applied adhesive 96 on the body side 88 of the web 90, suitable for prolonged contact to human skin and sufficiently adherent to removably hold the undergarment in place during use.

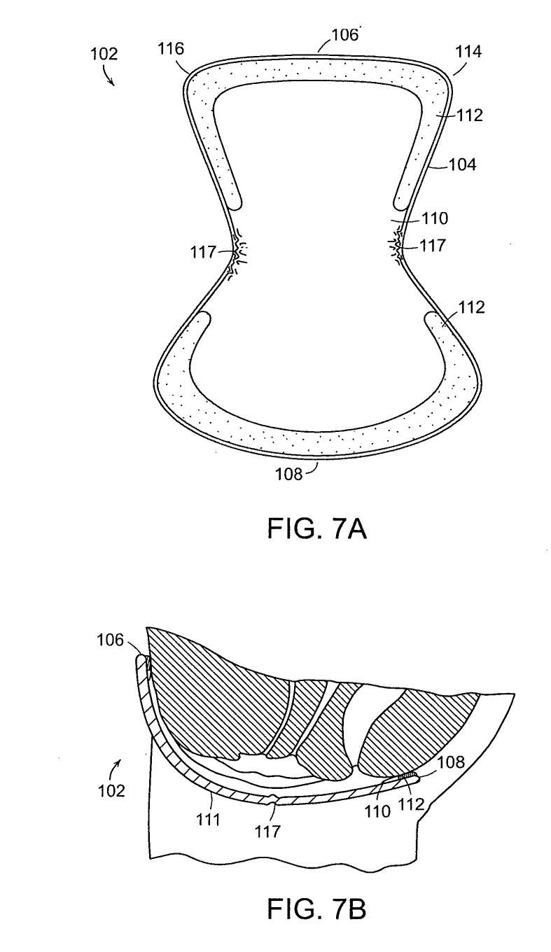

FIGS. 7A and 7B show another embodiment of the feminine undergarments disclosed here, which is longitudinally asymmetrical, that is, asymmetrical about a laterally extending line. Alternative embodiments having an adhesive tab extending only to the left or right or having non-identical left side and right side tabs (e.g., differently shaped or sized or a different number of tabs) would be both longitudinally and laterally asymmetrical. The embodiment of FIGS. 7A and 7B is sized and configured to cover, in use, an adult female wearer's vulva and to extend from the wearer's buttocks to or just below the navel. Undergarment 102 comprises a strapless, coreless web 104 having anterior end 106, posterior end 108, body side 110 and outside surface 111. In this embodiment, the anterior end 106 of the web 104 is wider (i.e., has a larger lateral dimension) than that of the embodiment of FIG. 1A. Also, the posterior end 108 of the web 104 is wider than the posterior edge of the embodiment of FIG. 1A. The undergarment 102 has pre-applied adhesive 112 on the body side 110 of the web 104, suitable for prolonged contact to human skin and sufficiently adherent to removably hold the undergarment in place during use. The periphery 114 of the web 104 has edge treatment 116 to control fraying and optionally in a duo-layer embodiment to join the two layers of the web together. The treatment 116 is different at the anterior end 106 and posterior end 108 than elsewhere. The treatment of the anterior end 106 and posterior end 108 are smooth to facilitate a smooth appearance under clothing, while the treatment elsewhere includes gathering 117 of the web fabric to provide a degree of preconfigured concave shape to the longitudinal center area of the web 104. From the disclosure and discussion above, it will be understood by those of ordinary skill in the art, that alternative embodiments may or may not include such edge treatment or different edge treatment.

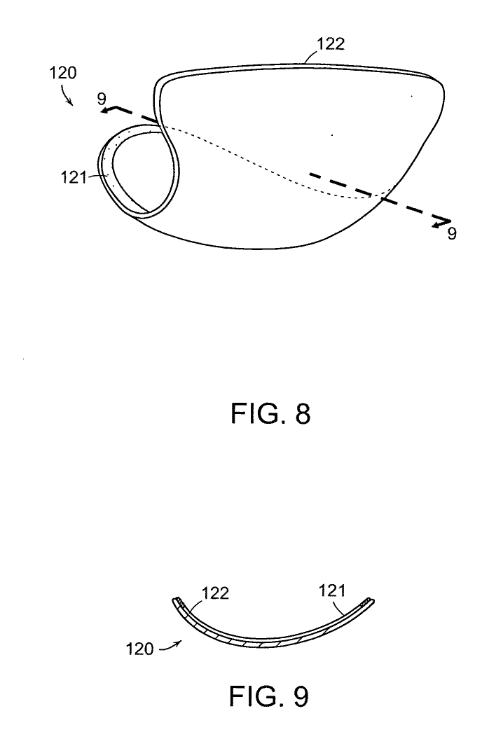

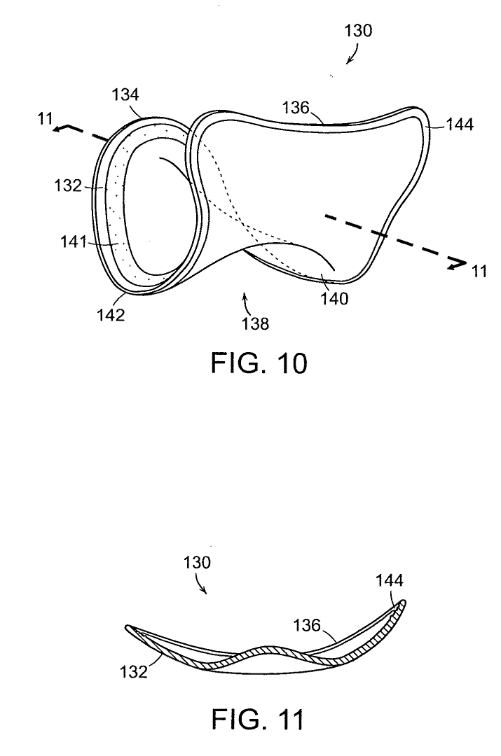

FIGS. 8 and 9 show another embodiment of the feminine undergarments disclosed here, specifically, an undergarment 120 wherein the web 122 has pre-applied adhesive 121 and is preconfigured to have a cup shape, i.e., to be concave toward the wearer (concave upwardly as oriented in FIG. 8). FIGS. 10 and 11 show another embodiment of the feminine undergarments disclosed here, specifically, an undergarment 130 wherein the web 132 is preconfigured to have a saddle shape. The web 132 with adhesive 141 is overall concave toward the wearer between the anterior and posterior ends 134, 136 and is convex in the central area 138 between right side 140 and left side 142. Peripheral treatment 144 of the web 132 is used in part to achieve the preconfigured shape.

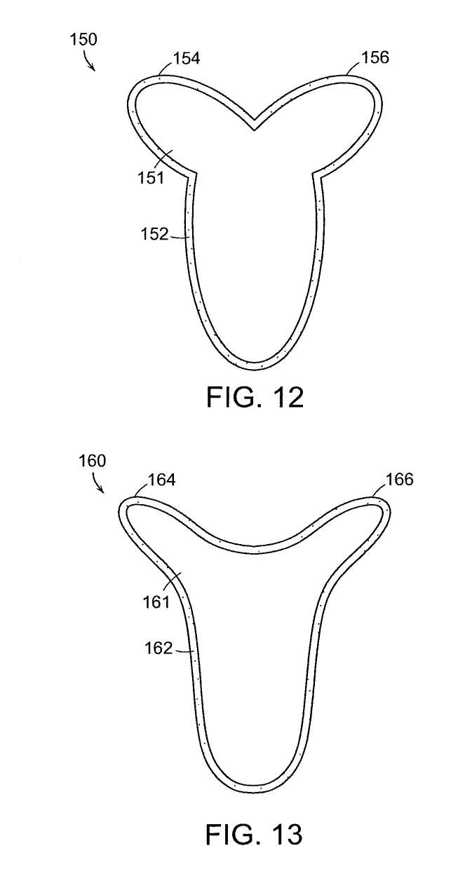

FIG. 12 shows another embodiment of the feminine undergarments disclosed here, specifically, an undergarment 150 wherein the web 151 has pre-applied adhesive 152 around its entire perimeter, including the perimeter of tabs or extensions 154 and 156.

FIG. 13 shows another embodiment of the feminine undergarments disclosed here, specifically, an undergarment 160 wherein the web 161 has pre-applied adhesive 162 around its entire perimeter, including the perimeter of angled tabs or portions 164 and 166.

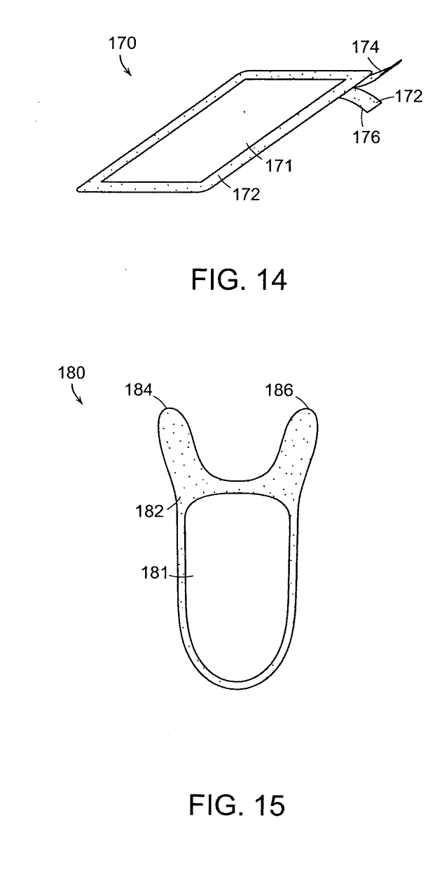

FIG. 14 shows another embodiment of the feminine undergarments disclosed here, specifically, an undergarment 170 wherein the parallelogram web 171 has pre-applied adhesive 172 around its entire perimeter and on asymmetrical tabs 174 and 176.

FIG. 15 shows another embodiment of the feminine undergarments disclosed here, specifically, an undergarment 180 wherein the web 181 has pre-applied adhesive 182 around its entire perimeter and on generally longitudinally extending tabs 184 and 186.

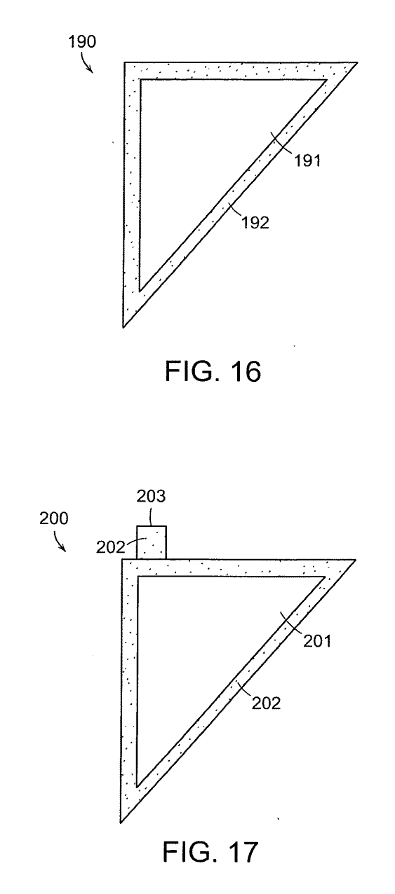

FIG. 16 shows another embodiment of the feminine undergarments disclosed here, specifically, an undergarment 190 wherein the triangular web 191 has straight sides and pre-applied adhesive 192 around its entire perimeter.

FIG. 17 shows another embodiment of the feminine undergarments disclosed here, specifically, an undergarment 200 wherein the triangular web 201 has pre-applied adhesive 202 around its entire perimeter and on tab 203.

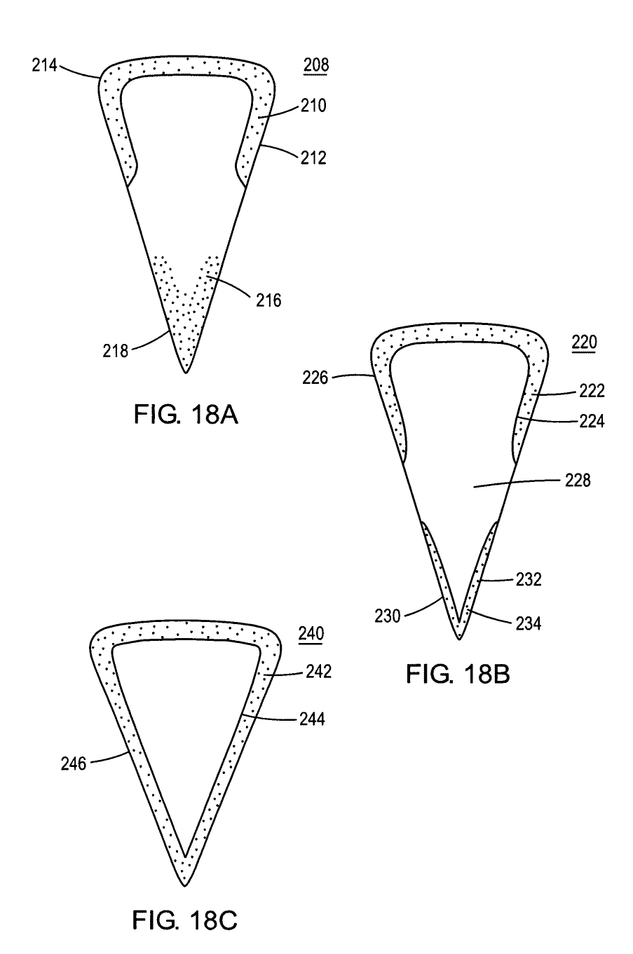

The embodiment of FIG. 18A is an undergarment 208 having adhesive 210 on edge treatment 212 along the perimeter of an anterior or forward portion 214 that is configured and sized to cover at least the vulva. It has overall adhesive 216 on a posterior portion 218 to directly contact and adhere to the anus and surrounding tissue of the wearer. Thus, the posterior portion of the embodiment of FIG. 18A would be normally out of sight, seated in the gluteal crease between the buttock cheeks at the anus. The undergarment 220 of the embodiment of FIG. 18B has adhesive 222 on edge treatment 224 along the perimeter of anterior portion 226 and posterior portion.

As in the embodiment of FIG. 18A, the web 228 of the embodiment of FIG. 18B is configured and sized to cover at least the vulva. Posterior portion 230 of the embodiment of FIG. 18B has adhesive 232 on edge treatment 234 along the perimeter. The posterior portion of the embodiment of FIG. 18B is sized and configured to be positioned close to, directly over, and/or completely covering the anus with adhesion to surrounding tissue, and to be normally invisible when viewed from the behind the wearer.

The undergarment 240 of the embodiment of FIG. 18C has adhesive 242 on edge treatment 244 along the entire perimeter of the web 246. As in the embodiments of FIG. 18A and FIG. 18B, the web of the embodiment of FIG. 18C is configured and sized to cover at least the vulva and the anus, with adhesion in this case to tissue surrounding the anus. Alternatively, certain embodiments of the undergarments generally as illustrated in FIGS. 18A-C may be configured to be worn with the pointed end as the anterior.

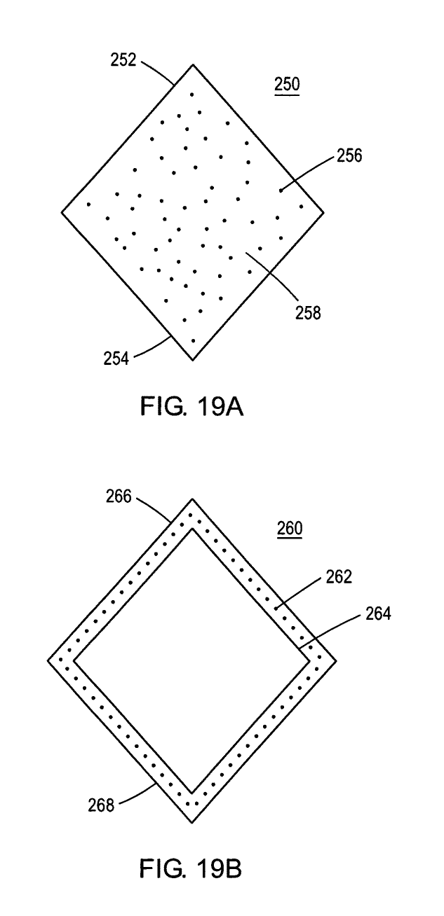

The undergarment 250 of the embodiment of FIG. 19A has anterior point 252 and posterior point 254, with adhesive 256 over most of the inside (i.e., body-side) surface of the web 258 to directly contact and adhere to at least the wearer's vulva. The diamond-shaped undergarment embodiment 260 illustrated in FIG. 19B has adhesive 262 on edge treatment 264 along the entire perimeter. It has anterior point 266 and posterior point 268. Embodiments according to FIGS. 19A and 19B can be sized to cover only the vulva or also additional tissue surrounding the vulva. Certain exemplary diamond-shaped embodiments can be sized to also cover the wearer's anus, optionally being more elongated from front to back than the illustrated embodiments.

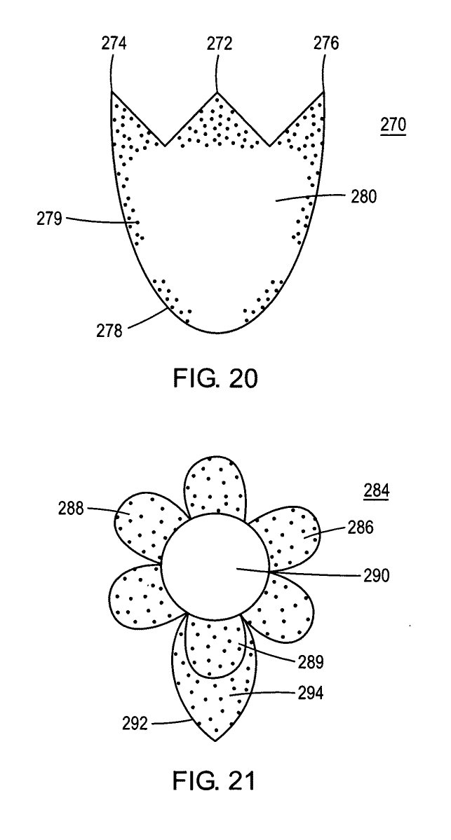

The undergarment embodiment 270 of FIG. 20 has central anterior point 272, side anterior points 274, 276 and posterior end 278. Alternatively, certain tulip-shaped embodiments of the undergarments disclosed here may be configured to be worn with the multi-pointed end to the posterior. Adhesive 279 is distributed around the periphery of the inside surface of web 280. The embodiment of FIG. 20 is configured to cover at least the wearer's vulva and, optionally, can be sized to also cover the wearer's anus, optionally being more elongated from front to back than the illustrated embodiments.

The undergarment 284 of FIG. 21 has adhesive 286 coating petal portions 288 extending radially from central portion 290 which does not carry adhesive. In alternative versions of daisy-petal shaped undergarments in accordance with the present disclosure, the petals optionally have edge treatment, as described above, and optionally have adhesive on the central web area. The embodiment of FIG. 21 is configured to cover the wearer's vulva and, in addition, has a posterior portion 292 extending rearward from petal 289. The extending posterior portion is configured and sized to cover the wearer's anus, optionally being more elongated from front to back than the illustrated embodiment. Posterior portion 292 in the illustrated embodiment has overall adhesive 294 on the body-side to directly contact and adhere to the anus and, in some embodiments also surrounding tissue. It will normally be out of sight, seated in the gluteal crease proximate the anus. In alternate versions of the embodiment of FIG. 21 the posterior portion optionally may have edge treatment along its perimeter and optionally may have adhesive only on such edge treatment. Alternatively, certain embodiments of the undergarments disclosed here may be configured to be worn with the pointed end to the anterior.

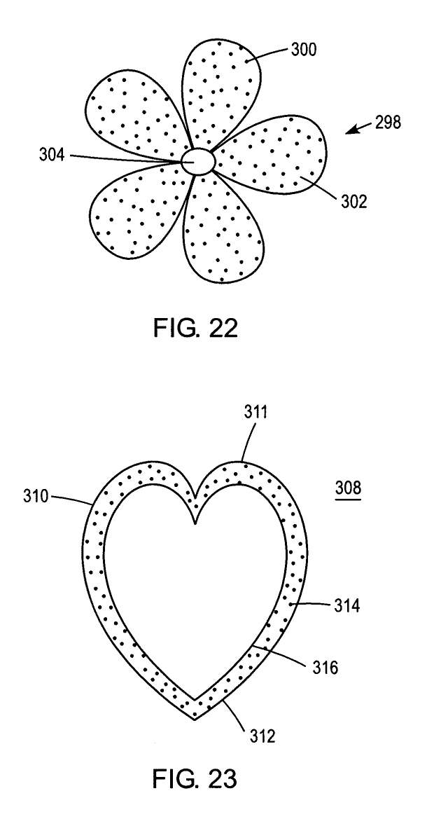

The undergarment 298 of FIG. 22 has adhesive 300 coating petal portions 302 extending radially from central portion 304 of the web, which does not carry adhesive. In alternative versions the petals optionally have edge treatment, as described above, and optionally may have adhesive only on the edge treatment. In alternative versions the central portion optionally may have adhesive on the body side surface. The embodiment of FIG. 22 is configured to cover at least the wearer's vulva. Alternative versions may optionally be configured and sized to cover the wearer's anus, optionally being more elongated from front to back than the illustrated embodiment. Alternative version may optionally have one petal elongated to cover the wear's anus.

The undergarment embodiment 308 of FIG. 23 has anterior lobes 310, 311 and posterior end 312. Alternatively, certain heart-shaped embodiments of the undergarments disclosed here may be configured to be worn with the two-lobed end to the posterior. The embodiment of FIG. 23 is configured to cover at least the wearer's vulva, and may optionally be sized to also cover the wearer's anus, optionally being more elongated from front to back than the illustrated embodiment. The embodiment of FIG. 23 has adhesive 314 distributed on edge treatment 316 extending along the entire perimeter of the web 318. In alternative versions, all or at least additional area(s) of the web may carry adhesive.

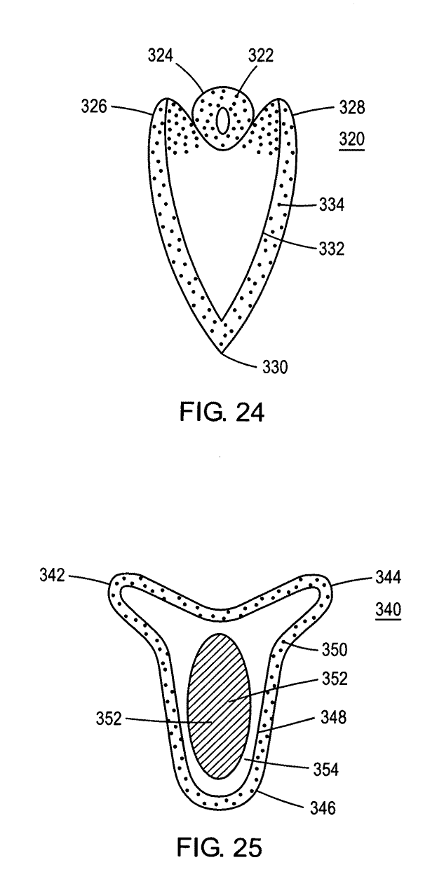

The undergarment 320 of FIG. 24 has a decorative circle 322 at the posterior end 324, along with lobes 326 and 328, and posterior point 330. Edge treatment 332 extending along the right and left sides of the web from the posterior point 330 toward lobes 326, 328 carry adhesive 334. Adhesive is carried as well on the anterior lobes. The embodiment of FIG. 24 is sized and configured to cover at least the wearer's vulva and, optionally, can be sized to also cover the wearer's anus, optionally being more elongated from front to back than the illustrated embodiments. Alternatively, certain embodiments of the undergarments disclosed here may be configured to be worn with the pointed end to the anterior. In alternative versions, all or at least additional area(s) of the web may carry adhesive.

The undergarment 340 of FIG. 25 has anterior lobes 342, 344 and posterior end 346, with full-circumference edge treatment 348. Alternative embodiments optionally have no edge treatment or have edge treatment along only one or more portions of the perimeter. In the illustrated embodiment, adhesive 350 is distributed on the edge treatment. The embodiment of FIG. 25 also comprises a removable and replaceable web component 352. It will be understood by those of ordinary skill in the art, that removable and replaceable web components as now described for the embodiment of FIG. 25 may optionally be incorporated into any of the other embodiments of the undergarments disclosed here. The embodiment of FIG. 25 is sized and configured to cover at least the wearer's vulva and, optionally, can be sized to also cover the wearer's anus, optionally being more elongated from front to back than the illustrated embodiments. In alternative versions, all or at least additional area(s) of the web may carry adhesive.

Removable and replaceable web component 352 is sized and configured to cover at least the wearer's vaginal opening. In certain exemplary embodiments it is sized and configured to cover most or all of the vulva and, optionally, surrounding tissue. In certain exemplary embodiments, the removable and replaceable web component may be configured to extend into and/or mold into some or all of the female genitilia (vaginal opening, vaginal canal, urethral opening, fill into the clitoris, clitoral hood). and/or anus. Web component 352 may be formed as a separate piece that can be held in position by an outer layer of the web 354 during use. It may be held in position in any suitable fashion, e.g., by a pocket formed in the web, by adhesive, by simply being captured in position, by other suitable technique or a combination of any of these. In embodiments wherein the web provides a pocket to hold the removable and replaceable web component in position during use, the pocket can be a simple concavity preformed in the web, a stitched envelope, etc. The removable and replaceable web component can be formed of any suitable material. For example, it may be formed of an open or closed cell foam, e.g., a sponge material, and/or gel, such as silicone gel, hydrocolloid gel, and/or other types of gel. In certain exemplary embodiments web component 352 can be a washable and reusable gel, foam, etc. sized and configured to simulate or enhance the shape or size of the vulva. In certain embodiments, the web may conform, mold, plug in, and/or fill-in the wear's vaginal opening, vulva, and/or anus. Certain embodiments may be comprised of more than one removable web component so that there is a separate web component for the vaginal opening, vulva, and/or anus. The separate web components may be attached to themselves, the web, and/or the periphery as described above. Optionally, it is washable and reusable. Optionally it can be impregnated with any one or more of the active agents described elsewhere in this disclosure. It will be understood by those of ordinary skill in the art, that removable and replaceable web components as now described for the embodiment of FIG. 25 may optionally be incorporated into any of the other embodiments of the undergarments disclosed here and may alternatively be non removable and/or non-replaceable in certain embodiments.

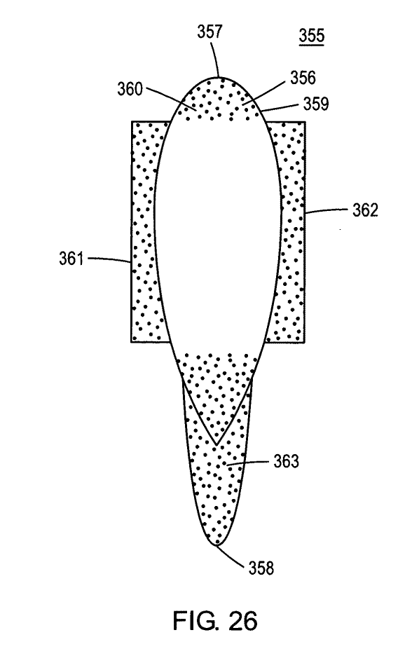

FIG. 26 illustrates an undergarment 355 having a strapless, coreless web 356 with anterior end or edge 357 posterior edge 358, body side 359 and outside (not seen). The web 356 is sized and configured to cover at least minimally, in use, a female wearer's vulva, extending from the bottom of the wearer's mons pubis to the perineum. More particularly in this embodiment, the web is more narrow laterally than the embodiment of FIG. 1a, but similar to the embodiment of FIG. 6. This embodiment has right-side 361 and left-side 362 longitudinal tabs between the anterior end 357 and the posterior end 358. The undergarment may optionally have an anus extension 363, to cover, in use, the wearer's anus. The undergarment 355 has pre-applied adhesive 360 on the body side 359 of the web; the longitudinal tabs 361,362; and the anus extension 363; suitable for prolonged contact to human skin and sufficiently adherent to removably hold the undergarment in place during use.

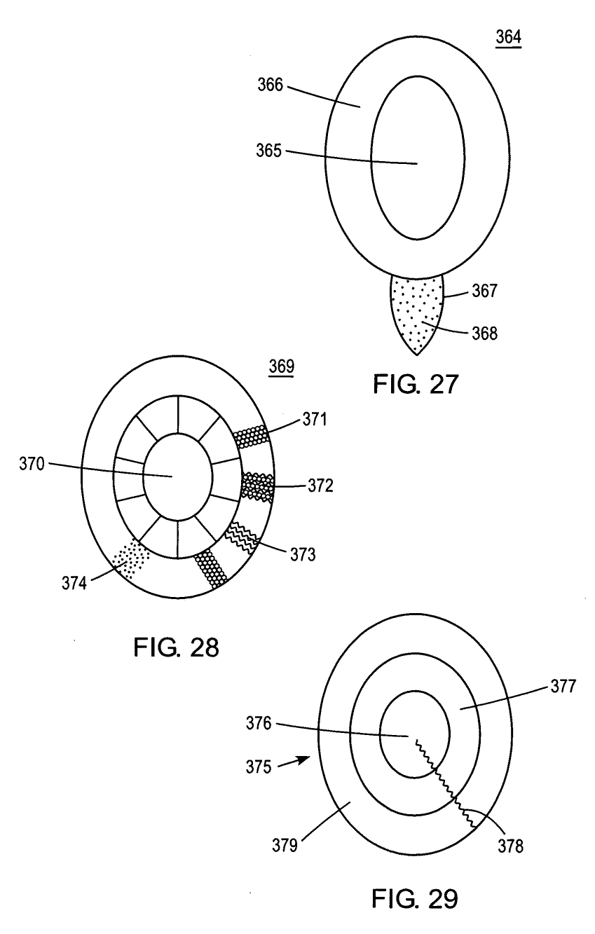

FIG. 27 illustrates an undergarment 364 wherein the undergarment constitutes an inner web 365 that may be a transdermal system (with or without active agent), “fill-in”, “mold”, and/or removable web. Optionally, certain embodiments, may have 365 extend to the entire undergarment (not shown). The perimeter web 366 may vary in diameter and may function as a suction device, have pre-applied adhesive (not shown), have ridges, dimples, grooves, ditches, skin glue (none of which shown), or have a combination of one or more these. Optionally, the undergarment may have an anus extension 367 with preapplied adhesive 368.

FIG. 28 illustrates an undergarment 369 wherein the web 370 is made to cup the wearer. In embodiment 369 the web is a suction device which optionally may consist of dimples 371, imprints 372, ridges 373, pre applied adhesive 374 or a combination of one or more which may be present in at least part of the undergarment. It is preconfigured to have a cup shape, i.e., to be concave toward the wearer (concave upwardly as oriented in FIG. 8).

FIG. 29 illustrates an embodiment 375 that is compatible meaning compatible with inserting and/or removing objects into the vulva 376. The web portion 376 may be removable, replaceable or left open. Optionally a second web portion 377 may be incorporated. The secondary web may be removable, replaceable, adhere to web 376. Webs 376 377, and 379 may be resized and/or incorporated together. The embodiment may optionally have less than three webs. The embodiment may have an outside periphery web 379 comprised of suction and/or adhesive (not shown). Optionally, the embodiment may have dimples 371, imprints 372, ridges 373, pre applied adhesive 374 or a combination of one or more which may be present in at least part of the undergarment. The embodiment may have a slit 378 to open the undergarment and reclose the undergarment. The slit 378 may be present in at least some of the undergarment. Optionally embodiment 375 may be resized and reconfigured to cover at least in part a human anus.

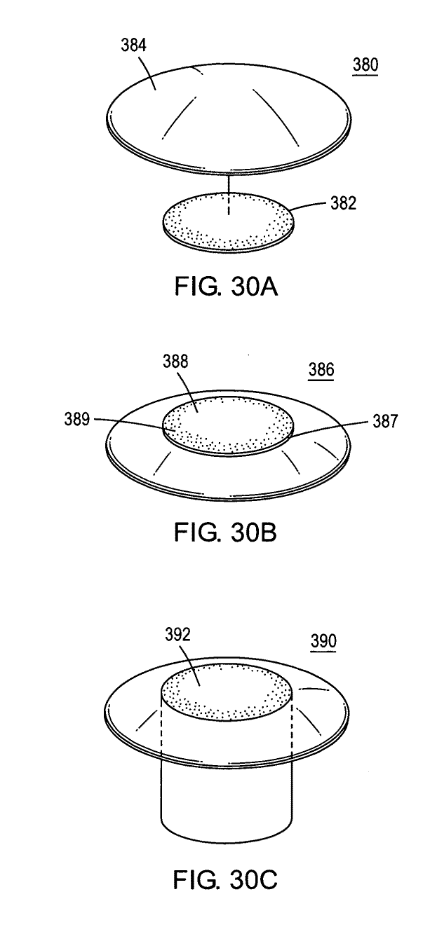

FIG. 30A illustrates an embodiment 380 that suctions to the wearers genial area. The embodiment may also have adhesive, grooves, ditches, ridges, imprints, or a combination thereof (not shown). The web component, 384, may be reusable, washable, fashionable, or a combination thereof. An alternate web 382 may be attached to the main web, 384. Alternately, the secondary web, 382, may be disposable after each application. Alternatively, the figure may be elongated or reconfigured to extend past the peritoneum and/or over the anus (not shown).

FIG. 30B, similar in concept to 30A, illustrates an alternate embodiment, 386. It may have an inside pocket or similar structure, 387, that may attach and facilitate the removal of the secondary web, 388. In addition, the secondary web, 388, may be removed for insertion of objects into the vulva. The secondary web may be held in place by adhesive, 389, or any other suitable means.

FIG. 30C, is another embodiment, 390, that suctions to the wearer's vulva. It has a removable and insertable secondary component that may extend into the vulva, 392.

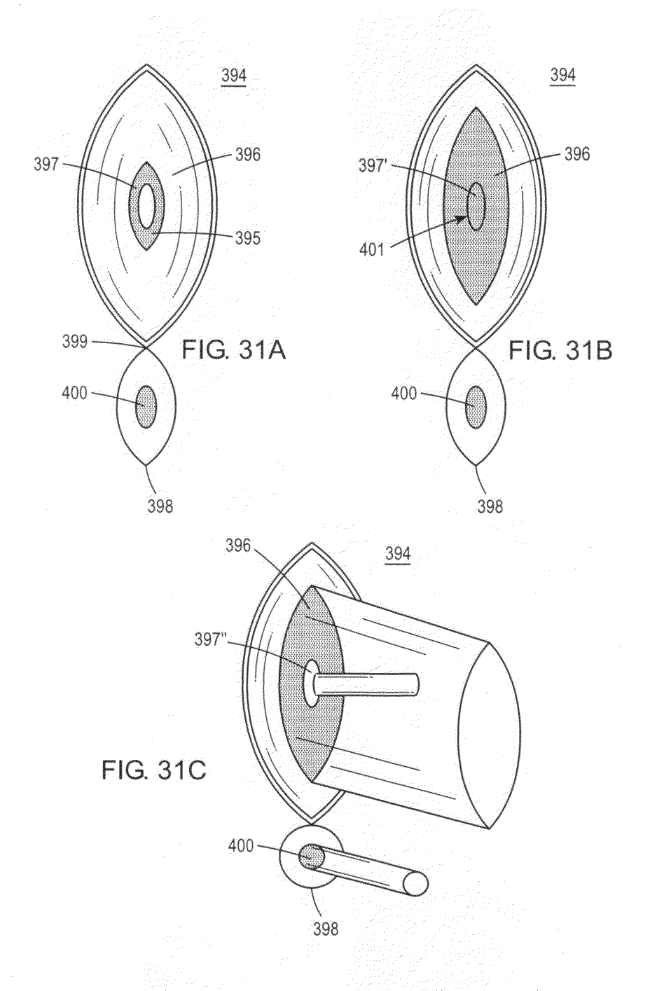

FIG. 31A, is another embodiment, 394, having a web that may conform to the female genital area, 396. It may adhere by suction, adhesive, grooves, ditches, imprints, ridges, or combination thereof (not shown). It has a secondary web component, 397, that may line, cover, seal, provide access to, insert into, mold to, conform to, extend into, fill into, or a combination thereof to the female vulva. It may comprise a third web component, 400, that may line, cover, seal, provide access to, insert into, mold to, conform to, extend into, fill into, or a combination thereof to the anus, anus opening, or a combination thereof. The embodiment may have a perforation or other means of separation, 399, to detach structure 398, from the main web, 395. Each structure may be used separately or in combination.

FIGS. 31B-31C are alternate versions of FIG. 31A showing, but not limited to, different depths of the secondary web component, 397′, 397″. Additionally FIG. 31B shows an alternate web component, 401, that covers, seals, fills into, molds into, molds over, or any combination thereof any opening and/or other access area that may be part of the product design and/or a result of product function. This alternate web component, 401, may also form a barrier, seal, cover, or any combination thereof, with any objects that are inserted into the opening, including but not limited to objects that are fully inserted in the opening (e.g. suppository) or protruding outward (e.g. urine catheter). Alternately, 397″ shows an alternate version that may allow for an opening if desired or required by the wearer.

As noted above, certain embodiments of the feminine undergarments disclosed here are disposable. Disposable versions of the undergarments can be formed of less durable or less costly materials. Disposable versions of the undergarments can be formed, for example, of biodegradable materials to be suitable for disposal into a municipal sewer or residential septic system. Such disposable versions are particularly useful for travel. Also, disposable as well as non-disposable versions can be advantageously used to control the spread of vaginal and/or anal secretion or discharge (e.g., mucus secretions, yeast infections, etc.) and vaginal infections from clothing to clothing and/or person to person. Such disposable versions can also, therefore, decrease washing expenses and time for individuals cleaning traditional undergarments that have vaginal and/or anal secretions. Additional uses for disposable versions of the undergarments will be apparent in view of this disclosure.

In any particular embodiment or version of the feminine undergarments disclosed here, the configuration of the strapless, coreless web may be longitudinally symmetric or asymmetric. Likewise, any such web may be laterally symmetric or asymmetric. Thus, for example, in certain exemplary embodiments the web may be altogether asymmetric. It may be both laterally and longitudinally asymmetric, and non-limiting examples of such webs have been discussed above. Longitudinally asymmetric embodiments may, for example, be wider at the anterior edge than at the posterior edge or wider at the posterior edge than at the anterior edge, etc. Other asymmetric embodiments may instead or in addition have laterally and/or longitudinally extending body-side adhesive tabs behind the midpoint between the anterior and posterior ends of the undergarment, or forward of the midpoint. Additional longitudinally asymmetric embodiments will be apparent to those of ordinary skill in the art given the benefit of this disclosure.

Although the present invention has been described above in terms of certain exemplary embodiments, it should be understood that other embodiments, other uses, alterations and modifications thereof within the principles of the invention will be apparent to those skilled in the art given the benefit of this disclosure. Such other embodiments, other uses, alterations and modifications thereof are intended to be within the scope the following claims. Also, it is intended that any and all of the various features, elements and details of the exemplary embodiments described above can be combined with each other and/or are interchangeable with each other. For example, one or more features, elements and details of any embodiment described above may be combined with and/or interchanged with any suitable features, elements and details of any other embodiment. It is also intended that the following claims be read as covering such other embodiments, other uses, alterations and modifications thereof as fall within the true spirit and scope of the invention as defined by the claims. It should be understood that the use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc.

All of the above-mentioned references are herein incorporated by reference in their entirety to the same extent as if each individual reference was specifically and individually indicated to be incorporated herein by reference in its entirety.

I claim as my invention each and all of the following:

1. A feminine undergarment comprising a strapless, coreless web sized and configured to cover, in use, at least a wearer's vulva, wherein the web is pre-adherent to the skin of the wearer, suitable for contact with human skin and sufficiently adherent to removably hold the undergarment in place during use covering the wearer's vulva.

2. The feminine undergarment of claim 1 wherein the undergarment is packaged for storage or shipment and a body-side surface of the web carries pre-applied adhesive, is inherently adhesive to skin, or both.

3. The feminine undergarment according to claim 1 wherein the web comprises a washable and reusable silicone material that is inherently adhesive to human skin.

4. An undergarment comprising:
a strapless, coreless web having an anterior end, a posterior end, a body side and an outside, wherein the web is sized and configured between the anterior end and the posterior end to cover, in use, at least a wearer's vulva; and
pre-applied adhesive on the body side of the web, suitable for contact with human skin to removably hold the undergarment in place during use covering a female wearer's vulva.

5. The feminine undergarment according to claim 4 wherein the web comprises cloth, paper, silicone, hydrocolloid, or a combination of any of them.

6. The feminine undergarment according to claim 4 wherein the web comprises cotton, flax, rayon, polyester, nylon, spandex, sponge, foam, lycra, fabric, woven material, unwoven material, high twist cotton elastic cloth, fibers, thermoplastic film, polyurethane, gel, viscous medium, gauze, twill, burlap, hemp, weave, silk, wool, natural plant material, canvas, denim, natural materials, seaweed, algae, medical foam, or a combination of any of them.

7. The feminine undergarment according to claim 4 wherein the web comprises silicone, hydrocolloid, or a combination of both material and the pre-applied adhesive comprises a silicone, hydrocolloid, or a combination of both.

8. The feminine undergarment according to claim 4 wherein the web comprises a removable and replaceable web component in a pocket formed by the web.

9. The feminine undergarment according to claim 4 wherein the web is translucent, transparent, or skin colored.

10. The feminine undergarment according to claim 4 wherein the pre-applied adhesive comprises a first adhesive on at least a first area of the body-side surface of the web and a second adhesive, different from the first adhesive, on at least a second surface area of the web.

11. The feminine undergarment according to claim 4 wherein the pre-applied adhesive comprises cell adhesion molecules.

12. The feminine undergarment according to claim 4 wherein the web is diamond-shaped, hourglass-shaped, daisy petal-shaped, triangular, heart-shaped, star-shaped, rectangular, oval, or pear-shaped.

13. The feminine undergarment according to claim 4 wherein the undergarment is being worn and the strapless, coreless web extends from the anterior end positioned on the female wearer between the navel and the mons veneris to the posterior end positioned at the perineum.

14. The feminine undergarment according to claim 4 wherein the undergarment is being worn and the strapless, coreless web extends from the anterior end positioned on the female wearer between the navel and the mons veneris to the posterior end covering at least a portion of the buttocks.

15. The feminine undergarment according to claim 14 wherein at least a portion of the posterior end of the strapless, coreless web is pre-configured to be body-side concave to follow the contours of the buttocks.

16. The feminine undergarment according to claim 4 wherein at least a portion of the strapless, coreless web is pre-configured to follow the contours of a human adult female's vulva.

17. The feminine undergarment according to claim 4 wherein the undergarment is being worn and the strapless, coreless web extends from the anterior end positioned on the female wearer at the mons veneris to the posterior end positioned at the perineum.

18. The feminine undergarment according to claim 4 wherein the undergarment is being worn and the strapless, coreless web extends from the anterior end positioned on the female wearer at the mons veneris to the anterior end covering at least a portion of the buttocks.

19. The feminine undergarment according to claim 18 wherein at least a portion of the posterior end of the strapless, coreless web is pre-configured to be body side concave to follow the contours of the buttocks.

20. The feminine undergarment according to claim 4 wherein at least a portion of the strapless, coreless web between the anterior end and the posterior is pre-configured to be body side concave to cup the female wearer's vulva.

21. The feminine undergarment according to claim 4 wherein at least a portion of the strapless, coreless web is pre-configured to be body side laterally convex.

22. The feminine undergarment according to claim 4 wherein the strapless, coreless web comprises at least a first extension between the anterior end and the posterior end, carrying at least a portion of the pre-applied adhesive.

23. The feminine undergarment according to claim 22 wherein the strapless, coreless web further comprises at least a second extension between the anterior end and the posterior end, carrying at least a portion of the pre-applied adhesive which may extend oppositely to the first extension.

24. The feminine undergarment according to claim 23 wherein the first and second extensions are sized and positioned to be adhered at least in part to the right and left thigh, respectively, of the wearer.

25. The feminine undergarment according to claim 23 wherein the first and second extensions are sized and positioned to be adhered at least in part to the buttocks, pubic bone, thigh, vulva, peritoneum, or combination thereof of the wearer.

26. The feminine undergarment according to claim 4 wherein the strapless, coreless web is a mono-layer web.

27. The feminine undergarment according to claim 4 wherein the strapless, coreless web is a multi-layer web.

28. The feminine undergarment according to claim 27 wherein the strapless, coreless web is a duo-layer web.

29. The feminine undergarment according to claim 27 wherein the strapless, coreless web is a tri-layer web.

30. The feminine undergarment according to claim 4 wherein the strapless, coreless web comprises a non-permeable layer.

31. The feminine undergarment according to claim 4 wherein the feminine undergarment is bio-occlusive.

32. The feminine undergarment according to claim 4 wherein at least a portion of the feminine undergarment is biodegradable.

33. The feminine undergarment according to claim 4 wherein at least a portion of the feminine undergarment is hypoallergenic.

34. The feminine undergarment according to claim 4 wherein at least a portion of the feminine undergarment is all-natural.

35. The feminine undergarment according to claim 4 wherein at least a portion of the feminine undergarment is organic.

36. The feminine undergarment according to claim 4 wherein the feminine undergarment is suitable for prolonged contact with human skin.

37. The feminine undergarment according to claim 4 wherein the strapless, coreless web is permeable.

38. The feminine undergarment according to claim 4 wherein the strapless, coreless web is non-permeable.

39. The feminine undergarment according to claim 4 wherein the strapless, coreless web is semi-permeable.

40. The feminine undergarment according to claim 4 wherein the strapless, coreless web comprises at least a layer of woven, unwoven, knitted, lace, stitched, or pressed fabric or a combination of any of them.

41. The feminine undergarment according to claim 4 wherein at least a portion of the strapless, coreless web is sufficiently elastically stretchable to cup, in use, the female wearer's vulva.

42. The feminine undergarment according to claim 4 wherein the strapless, coreless web is seamless.

43. The feminine undergarment according to claim 4 wherein the strapless, coreless web shows a decorative image, shape, print, imprint, or combination thereof.

44. The feminine undergarment according to claim 4 wherein the removable protective layer is a sealed envelop, sheet, pouch, case, lid, continuous piece, at least one or more pieces, wrapper, continuous piece consisting of an inner and outer layer, biodegradable layer, cloth layer, or combination thereof enclosing the strapless, coreless web, web insert, pre-applied adhesive or a combination thereof.

45. The feminine undergarment according to claim 4 wherein the pre-applied adhesive forms a continuous adhesive zone on the periphery of the body side of the strapless, coreless web.

46. The feminine undergarment according to claim 4 wherein the pre-applied adhesive is positioned intermittently about the periphery of the body side of the strapless, coreless web.

47. The feminine undergarment according to claim 4 wherein the pre-applied adhesive covers the entire body side of the strapless, coreless web.

48. The feminine undergarment according to claim 4 wherein the pre-applied adhesive covers less than the entire body side of the strapless, coreless web.

49. The feminine undergarment according to claim 4 wherein the pre-applied adhesive covers at least a portion of a laterally extending anterior edge of the strapless, coreless web.

50. The feminine undergarment according to claim 4 wherein the pre-applied adhesive covers at least a portion of a laterally extending posterior edge of the strapless, coreless web.

51. The feminine undergarment according to claim 4 wherein the pre-applied adhesive covers at least the entire laterally extending anterior edge of the strapless, coreless web.

52. The feminine undergarment according to claim 4 wherein the pre-applied adhesive covers at least the entire laterally extending posterior edge of the strapless, coreless web.

53. The feminine undergarment according to claim 4 further comprising at least one extending adhesive tab.

54. The feminine undergarment according to claim 4 further comprising a symmetrical pair extending tabs, each carrying adhesive positioned for contact with a corresponding one of the inner thighs of a wearer.

55. The feminine undergarment according to claim 4 further comprising asymmetrical pair of extending tabs, each carrying adhesive positioned for contact with the wearer's skin.

56. The feminine undergarment according to claim 4 wherein the strapless, coreless web; web insert; pre-applied adhesive; or a combination thereof are all-natural.

57. The feminine undergarment according to claim 4 wherein the strapless, coreless web is longitudinally asymmetric.

58. The feminine undergarment according to claim 4 wherein the strapless, coreless web is laterally asymmetric.

59. The feminine undergarment according to claim 4 further comprising, inherently containing, impregnated, coated with, delivers, or a combination thereof an active agent.

60. The feminine undergarment according to claim 59 wherein the active agent comprises a perfume, cologne, essential oils, ions, flavorant, pharmaceutical, nutriceutical, herbaceutical, nutrient, phytonutrient, probiotic, anti-viral, viricide, antibacterial, antiperspirant, anti-fungal, antiseptic, deodorant, disinfectant, odor neutralizer or suppressant, anti-microbial, microbicide, anti-itching agent, desiccant powder or other drying agent, cooling agent, vitamins, antioxidant, skin rejuvenating agent, pH stabilizing agent, libido enhancement and/or stimulant, hormonal balancing, sun protectant, lubrication, medical foam, medical gel, medical sponge, or a mixture of any two or more of them.

61. The feminine undergarment according to claim 59, wherein the active agent is provided by the web, the web insert, the pre-applied adhesive, or a combination thereof to contact the wearer's skin, genital area, vaginal opening, urethral opening, anal opening, gluteal area, buttocks, or a combination thereof.

62. The feminine undergarment according to claim 4 further comprising a transdermal system comprising an active agent in a time-release matrix positioned to contact the wearer's skin, vaginal opening, urethral opening, anal opening, or any combination thereof.

63. The feminine undergarment according to claim 62 wherein the active agent, comprises perfume, cologne, essential oils, flavorant, pharmaceutical, nutriceutical, herbaceutical, nutrient, phytonutrient, probiotic, anti-viral, viricide, antibacterial, antiperspirant, anti-fungal, antiseptic, deodorant, disinfectant, odor neutralizer or suppressant, anti-microbial, microbicide, anti-itching agent, desiccant powder or other drying agent, cooling agent, vitamins, antioxidant, skin rejuvenating agent, pH stabilizing agent, libido enhancement and/or stimulant, hormonal balancing, sun protectant, lubrication, or a mixture of any two or more of them.

64. The feminine undergarment according to claim 62 which is configured to extend into the female genital area, urethral opening, vaginal opening, anal opening, or a combination thereof.

65. The feminine undergarment according to claim 4 wherein the strapless, coreless web is a multi-layer web, comprising an outer layer and an inner layer attached to the outer layer.

66. The feminine undergarment according to claim 65 wherein the inner layer is attached continuously at its periphery to the outer layer.

67. The feminine undergarment according to claim 65 wherein the inner layer is attached intermittently at its periphery to the outer layer.

68. The feminine undergarment according to claim 65 wherein the inner layer is attached only at its periphery to the outer layer.

69. The feminine undergarment according to claim 65 wherein the inner layer is attached to the outer layer by stitching, thermobinding, thermally, adhesively, or a combination thereof.

70. The feminine undergarment according to claim 4 which is medically approved, sterilized, or a combination thereof.

71. The feminine undergarment according to claim 4 having a protective layer, reinforcing layer, supporting layer, absorbing layer, encasing, or a combination thereof on the body side, outside, between removable webs, between layers, or a combination thereof.

72. The feminine undergarment according to claim 65 wherein the inner layer is smaller than the outer layer, the inner layer being sized and positioned to cover the wearer's vaginal opening, anal opening, or combination thereof.

73. The feminine undergarment according to claim 65 wherein the inner layer is larger than the outer layer, the inner layer being sized and positioned to extend from the anterior end positioned on the female wearer forward of the vulva to the posterior end positioned at or rearward of the perineum and the outer layer being sized to cover the wear's vulva, wearer's anus, or combination thereof.

74. The feminine undergarment according to claim 65 wherein at least a portion of the inner layer is natural material.

75. The feminine undergarment according to claim 65 wherein at least a portion of the inner layer is cotton.

76. The feminine undergarment according to claim 65 wherein at least a portion of the inner layer is synthetic material.

77. The feminine undergarment according to claim 65 wherein at least a portion of the inner layer comprises a combination of natural and synthetic materials.

78. The feminine undergarment according to claim 65 wherein at least a portion of the web is pre-configured at least in part by the attachment of the inner layer to the outer layer.

79. The feminine undergarment of claim 4 having suction means for holding the web in place during use covering a female wearer's vulva, anus, surrounding genital area, or combination thereof.

80. The feminine undergarment of claim 79 wherein the suction means comprises suction material pre-applied on the body side of the web, suitable for contact to human skin.

81. The feminine undergarment of claim 79 wherein the web is held in position on the wearer entirely by suction.

82. The feminine undergarment of claim 79 wherein the suction means comprises grooves, ridges, imprints, dimples, or combination thereof in the body-side surface of the web.

83. The feminine undergarment according to claim 4 further comprising a removable protective layer covering at least the adhesive and removable by the wearer prior to use of the undergarment.

84. The feminine undergarment according to claim 4 wherein the strapless, coreless web has one or more guides for size-adjustment of the web by the user.

85. The feminine undergarment according to claim 84 wherein the one or more guides for size-adjustment of the strapless, coreless web by the user comprises at least one line of perforations.

86. A package of feminine undergarments comprising multiple feminine undergarments and outer packaging containing at least the multiple feminine undergarments, wherein each of the multiple feminine undergarments comprises:
a strapless, coreless web having an anterior end, a posterior end, a body side and an outside, wherein the web is sized and configured between the anterior end and the posterior end to cover, in use, at least an adult female wearer's vulva;
pre-applied adhesive, pre-applied suction material or a combination of both on the body side of the web, suitable for prolonged contact to human skin and sufficiently adherent to removably hold the undergarment in place during use covering a female wearer's vulva; and
a removable protective layer covering at least the adhesive or suction material and removable by the wearer prior to use of the undergarment.

87. The package of feminine undergarments according to claim 86, wherein the strapless, coreless web of at least some of the feminine undergarments are pre-configured and are packaged in the outer packaging in a substantially flat condition; rolled in any acceptable fashion, folded in any acceptable fashion; conform to at least part of the pre-configured shape; conform to the shape of the intended application site; or any combination thereof.

88. The undergarment according to claim 4 that comprises an opening, slit, access point, re-closable opening, or combination thereof to facilitate insertion of devices, tampons, catheters, diagnostics, medicine, suppositories, probes, objects, or a combination thereof into the genital area, vaginal opening, urethral opening, anal opening, gluteal crease, or a combination thereof.

89. The feminine undergarment according to claim 4 wherein at least a portion is edible.

90. The feminine undergarment of claim 4 comprising ionic binding, grooves, ditches ridges, imprinting, adhesive, suction, dimples, or a combination thereof.

91. The feminine undergarment according to claim 4 wherein the web, web inserts, adhesive, removable protective barrier, or a combination thereof comprises a device to facilitate handling, removing, inserting, dispensing, storing, traveling, or combination thereof.

92. The feminine undergarment according to claim 4 wherein the web comprises a removable and replaceable web component that can be applied to a primary web or adhesive component.

93. The feminine undergarment according to claim 4 wherein the web, adhesive, removable web component, web insert, or combination thereof has at least one slit, opening, point of access, or a combination thereof to allow access to at least part of the female genital area, anus, urethra, or a combination thereof.

94. The feminine undergarment according to claim 4 wherein the web, adhesive, removable web, active agent, web insert, or combination thereof fills into at least some of the wearer's vulva, anus, surrounding site of application, or a combination thereof.

95. The feminine undergarment according to claim 4 wherein the web, adhesive, removable web, active agent, web insert, or combination thereof molds over the site of application.

96. The feminine undergarment according to claim 4 wherein the web, adhesive, removable web, active agent, web insert, or combination thereof molds into the site of application.

97. The feminine undergarment according to claim 4 that seals the genital area, vaginal opening, anal opening, or combination thereof.

98. The feminine undergarment according to claim 4 wherein all of the web, insertable web component, removable component, adhesive, or combination thereof is disposable.

99. The feminine undergarment according to claim 4 wherein the at least part of the web, insertable web component, removable web component, adhesive, or combination thereof is absorbent.

100. The feminine undergarment according to claim 4 wherein the at least part of the web, insertable web component, removable web component, adhesive, or combination thereof has a coating, film, layer, multiple layers, or combination thereof.

101. The feminine undergarment according to claim 4 wherein the at least part of the web, adhesive, removable component, insertable component, or combination thereof is hydrophilic, hydrophobic, hygroscopic, basic, acidic, neutral, or a combination thereof.

102. The feminine undergarment according to claim 4 wherein the at least part of the adherent property comprise adhesive, skin glue, rubber, acrylic, suction, ditches, tape, silicone, hydrocolloid, grooves, imprints, ridges, or combination thereof.

103. The feminine undergarment according to claim 4 that is at least partly washable, removable, disposable, reusable or a combination thereof.

104. The feminine undergarment according to claim 1 that lines into the genital area, vaginal opening, anal opening, or a combination thereof.

105. The feminine undergarment according to claim 4 may be sized to cover the vulva without extending either laterally or forward of the labia or optionally may extend rearward into the gluteal crease, with or without body-side adhesive on the gluteal crease portion of the web.

106. The feminine undergarment according to claim 4 that is adherent in active wearers.

107. The feminine undergarment according to claim 4 that may adhere to wet or moist skin.

108. A feminine undergarment according to claim 4 wherein at least part of the web, insertable web component, removable web component, or combination thereof, has a internal lining, member, device, extension, plug, or combination thereof that inserts into at least part of the wearer's genital area, vaginal opening, anal opening, urethral opening, or a combination thereof.

109. An undergarment comprising:
a strapless web having an anterior end, a posterior end, a body side and an outside, wherein the web is sized and configured between the anterior end and the posterior end to cover, in use, at least a wearer's genital area; and
pre-applied adhesive, suction, grooves, imprinting, dimples, ridges or a combination thereof on the body side of the web, suitable for contact with human skin to removably hold the undergarment in place during use covering the genital area
one or more insertable, removable, replaceable, medically configured, device, extension, or a combination thereof web component that may be configured to mold into, fill into, line, extend into, provide a flush surface, cover, seal, or any combination thereof to the genital area, anal area, vaginal opening, urethral opening, anal opening or combination thereof.

110. The undergarment according to claim 109 that has at least one reapplication.

111. The undergarment according to claim 109 that can be used in combination with other devices, equipment, diagnostics, medicines, components, active agents, or a combination thereof.

112. The sterile undergarment according to claim 109 that is configured to wearers who are burned, disfigured, traumatized, mutilated, immobile, or a combination thereof for medical use, fashion use, common use, accessory use, or a combination thereof.

113. An undergarment comprising:
a strapless web having an anterior end, a posterior end, a body side and an outside, wherein the web is sized and configured between the anterior end and the posterior end to cover, in use, at least a wearer's genital area; and
pre-applied adhesive, suction, grooves, imprinting, dimples, ridges or a combination thereof on the body side of the web, suitable for contact with human skin to removably hold the undergarment in place during use covering the genital area
one or more insertable, removable, replaceable webs, devices, extensions, or a combination thereof that extends into, configures to, molds into, fills into, lines, covers, seals, or a combination thereof to thereof to the genital area, anal area, vaginal opening, urethral opening, vaginal canal, anal opening or combination thereof.
opening, slit, access point, re-closable opening, or combination thereof to facilitate insertion of devices, tampons, catheters, diagnostics, medicine, suppositories, probes, objects, or a combination thereof into the genital area, vaginal opening, urethral opening, anal opening, gluteal crease, or a combination thereof.
